(19) United States
(12) Patent Application Publication
(10) Pub. No.: US 2010/0203535 A1
(43) Pub. Date: Aug. 12, 2010

(54) GENETIC ANALYSIS

(76) Inventors: Susan Anne Ross Stenhouse, Glasgow (GB); Victoria Murday, Glasgow (GB); Daniel Matthew Ellis, Glasgow (GB)

Correspondence Address: WINSTEAD PC, P.O. BOX 50784, DALLAS, TX, 75201, US

(21) Appl. No.: 12/675,206

(22) PCT Filed: Sep. 1, 2008
(86) PCT No.: PCT/GB2008/002938

§ 371 Date: Apr. 28, 2010

(30) Foreign Application Priority Data
Aug. 31, 2007 (GB) 0716947.7

(51) Int. Cl. C12Q 1/68; C07H 21/00

(52) U.S. Cl. 435/6; 536/24.33

(57) ABSTRACT

The present invention provides methods for excluding a gene as being involved in, associated with or causative of a genetic disorder in a family.

### FIELD OF THE INVENTION

The present invention provides methods for excluding a gene as being involved in, associated with or causative of a genetic disorder in a family.

### BACKGROUND

Over the last few years it has become apparent that many common inherited adult genetic disorders can occur as result of alterations in several different genes. Usually only one of the causative genes is altered in an individual family. If genetic testing is to be available for unaffected individuals within a family it is necessary to identify the specific gene implicated in that family and the individual mutation responsible. An example of this would be breast cancer (especially familial breast cancer) which can be caused by a mutation in one of two genes BRCA1 and BRCA2, but can also occur as a result of alterations in a number of other genes, TP53 and PTEN being examples. In addition 25% of familial breast cancer is currently genetically unaccounted for and so further genes are likely to be added to the list. Long QT, a condition predisposing to sudden cardiac death, is known to be caused by at least 8 different genes, and hypertrophic cardiomyopathy, a condition affecting 1 in 500 of the population and also associated with sudden cardiac death, has so far been shown to be caused by many different genes with 75% being due to mutations in 5 different genes.

The present invention provides methods which may allow for a reduction in the number of genes to be sequenced by between 50% and 80%, with a rapid high throughput technology. The methods described herein could eliminate up to 80% of sequencing for these disorders with consequent time and cost savings.

Genetic linkage refers to the situation where two loci lie so close to each other on the chromosome that they tend to be inherited together more often than would be expected by random segregation. The statistical distortion of random segregation is used to map both diseases and genes. If the location of one locus is known and is inherited with a disease more often than would be expected to by chance then the disease and locus are likely to lie close to each other on the same chromosome. This principle is also used in association studies in populations looking for susceptibility genes for complex disease.

Many genes responsible for rare single gene disorders were mapped and continue to be mapped by linkage. Mapped disease genes can be identified and sequenced by identifying potential genes within the region. Diagnostic molecular genetics laboratories can use linked marker loci known to track with a disorder to predict who in a family is affected. However the number of families to which this can be applied is small, as few are large enough or have enough living relatives. Multiple samples from related individuals in more than one generation are required to establish ‘phase’ of the disorder i.e. which marker allele tracks with the disorder in that family. In addition if a disorder is caused by more than one gene then linkage is unsuitable for diagnostic testing as the causative gene in each family needs to be established first.

More recently linkage studies using consanguineous families, have been used to map the genes responsible for a variety of recessive diseases. Often this type of linkage data can also exclude a candidate gene's involvement, since the affected individuals must be identical by descent and all markers must be homozygote in or around a disease gene. In addition exclusion mapping in subpopulations has been suggested as a method of excluding regions of the genome from containing susceptibility genes.

The object of the present invention is to obviate or mitigate at least one of the aforementioned problems.

### SUMMARY OF THE INVENTION

The present invention is based upon the principles of exclusion mapping, but is applied to specific loci known to be implicated in susceptibility. By demonstrating that affected individuals have no allele identical by descent at the susceptibility gene, it may be possible to exclude that gene as causative in affected individuals. By focussing on the presence of SNP alleles which are oppositely homozygous in affected subjects, the need to establish phase (i.e. on which chromosome the gene associated with, or causative of the genetic disorder is located) is eliminated. In turn the methods described herein may require just two affected and related subjects. This represents a considerable advantage over the prior art.

Thus, in a first aspect, the present invention provides a method of excluding the involvement of a gene in a genetic disorder in a family, said method comprising the steps of:
(a) selecting a population of single nucleotide polymorphisms (SNPs) that
   i. are proximate to the gene; and
   ii. have a minor allele frequency sufficient to establish an appropriate level of heterozygosity; and
(b) identifying said SNPs in nucleic acid samples provided by each of at least two subjects affected by the genetic disorder and linked by pedigree;

wherein, the presence of SNP alleles which are oppositely homozygous in at least two of the affected subjects, indicates that the gene is unlikely to be involved the genetic disorder.

It is to be understood that the phrase “excluding the involvement of a gene” may be taken to encompass the process of determining that a gene is not associated with and/or causative of a genetic disorder in a family.

The term “genetic disorder” may be taken to be any disease or condition which has a genetic aetiology—i.e. disorders in which the symptoms are caused or contributed to by one or more genes and/or associated nucleic acid sequences. It is to be understood that “associated nucleic acid sequences” may include, for example, promoter regions, transcription factor binding sites, enhancer elements and/or other associated regulatory elements involved (either directly or indirectly) with gene expression.

Typically, genetic disorders result from the presence of some form of abnormality, for example a mutation and/or alteration of the “wild type” nucleotide sequence which comprises the gene and/or an associated nucleic acid sequence. Accordingly, the methods described herein may be taken to relate to methods of excluding mutations or alterations in a particular gene as being associated with, or causative of, a genetic disorder in a family.

A mutation and/or alteration may modulate, for example, the activity and/or level of expression of a gene and/or its protein product. For example, a mutation or alteration in a gene sequence may result in an increase or decrease in the expression of the gene and/or its protein product. Alternatively, a mutation and/or alteration may result in the partial or total loss of a gene's (or its product's) function and/or activity. By way of example, mutations in the promoter region of a particular gene may modulate the activity and/or level of expression of that gene. Examples of mutations and/or alterations which may result in modulation of gene activity and/or expression, include single or multiple base pair insertions, inversions, substitutions and/or deletions. Accordingly, such mutations and/or alterations may be associated with a particular genetic disorder.

Genetic disorders may be regarded as either “dominant” or “recessive”. A dominant genetic disorder involves a gene or genes which exhibit(s) dominance over a normal (healthy) gene or gene's. As such, in dominant genetic disorders only a single copy of an abnormal gene is required to cause or contribute to, the symptoms of a particular genetic disorder. In contrast, recessive genetic disorders are those which require two copies of the abnormal/defective gene to be present.

One of skill in the art will appreciate that all subjects with any type of dominant genetic disorder may be subjected to the methods described herein. As such, and in one embodiment, the present invention may be used to a gene as being involved in, associated with, or causative of genetic disorders such as, for example familial Breast cancer (the term “breast cancer” as used herein should be taken to encompass familial breast cancer), Hereditary haemorrhagic telangectasia, Hereditary spastic paraplegia, Cerebral cavernous malformations, Hypertrophic cardiomyopathy, Dilated cardiomyopathy, Long QT, Adult polycystic kidney disease, Tuberous sclerosis, Spinocerebellar ataxia, Alzheimer's, Marfan syndrome, Noonan syndrome, Dominant retinitis pigmentosa, Multiple epiphyseal dysplasia, Ehlers Danlos, Hereditary colorectal cancer, Juvenile polyposis and/or Familial paraganglioma.

In one embodiment, the present invention concerns genes associated with or causative of familial breast cancer. In particular the invention may provide a method for excluding the involvement of the BRCA1 and/or BRCA2 genes in familial breast cancer in a family.

In a further embodiment, the present invention concerns genes associated with or causative of hypertrophic cardiomyopathy and/or dilated cardiomyopathy. In particular the invention may provide a method for excluding the involvement of one or more of the genes selected from the group consisting of TTN, MYH6/7, MYBPC3, RAF1, PRKAG2, TPM1, TNNT2, MYLK2, TNNI3, MYL3, MYL2 and/or CAV3 in instances of hypertrophic cardiomyopathy or dilated cardiomyopathy.

It is to be understood that the terms “family” and/or “linked by pedigree” may be taken to encompass a population of individuals related by blood. The present invention provides methods in which the selected SNPs are identified in nucleic acid samples provided by each of at least two (affected) subjects linked by pedigree. It is to be understood that the term “linked by pedigree” is intended to encompass subjects having a suitable relationship. Advantageously, those subjects linked by pedigree may be considered as members of the same family or as consanguineous relatives. While any form of blood relationship may be considered suitable, it is important to note that subjects representing parent/child pairs are not appropriate for use in this method as the data generated therefrom will always be uninformative. Accordingly, to minimise the generation of uninformative data, the pedigree links that exist between the at least two affected subjects should not represent, constitute or comprise parent/child links. One of skill in the art will appreciate that, since parent child pairs always have one allele in common—it is not possible for a parent and child to be oppositely homozygous for a particular SNP allele.

Advantageously, suitable relationships between affected subjects linked by pedigree may include, for example sibling, cousin and aunt/uncle/niece/nephew relationships.

One of skill in the art will understand that for more distant relatives (for example aunts/uncles/nieces/nephews and/or cousins) the chance of each individual comprising the relationship having inherited a common copy of a particular gene from a relative is lower than in more closely related individuals (for example siblings).

One of skill in the art will be familiar with the term “single nucleotide polymorphism” or “SNP”. Briefly, a “SNP” represents a form of variation in a genome wherein a particular nucleotide of the genome varies between members of a population. By way of example, a SNP may comprise two alleles (i.e. one of two possible nucleotides at a particular locus)—and in such cases, some of the individuals within a population may carry one SNP allele at a particular locus while others may carry the other allele at the same locus.

Within a population and at a particular locus, one SNP allele may occur less frequently than another or other SNP allele/alleles and as such it is possible to calculate the ratio of chromosomes within a population carrying the less frequently occurring SNP allele to those chromosomes carrying the more common allele. This ratio is known to those skilled in this field as the “minor allele frequency” (referred to hereinafter as the “MAF value”).

In one embodiment, and to maximise the likelihood that the information returned by the methods described herein is informative, it is preferable for the parents of each of the at least two affected individuals to be heterozygous for the alleles which comprise at least one of the selected SNPs. One of skill in the art will appreciate that the chance of this occurring depends upon the MAF value of each of the SNPs concerned and becomes more likely as the MAF value approaches 0.5.

As such, a MAF value sufficient to establish heterozygosity should be taken to mean a MAF value which indicates a high probability that, within a population, there are a large number of individuals who are heterozygous for the SNP alleles. In this way, by selecting SNPs with a MAF value sufficient to establish heterozygosity, there is a high probability that the parents of each of the at least two affected individuals selected for use in the methods described herein, will be heterozygous for the SNP alleles and that the method will yield informative data.

Preferably, the MAF value of each of the selected SNPs is greater than 0.1, preferably greater than about 0.2 and even more preferably greater than about 0.3.

In addition, the selected SNPs should be proximate to the gene known to cause (or be associated with) the genetic disorder. Advantageously, SNPs which are proximate to a gene may be those which are located within the gene as well as those which are adjacent to, associated with or linked to that gene. One of skill in the art will appreciate that only those SNPs which are proximate to a gene may be considered as associated with or linked to that gene.

Typically, a SNP which occupies a locus distant (or not proximate) to a particular gene, is less likely to be associated with or linked to that gene (i.e. the more distant the SNPs from the gene, the greater the chances of recombination). The skilled man will understand that what may be considered as proximate, linked or unlinked to a gene may vary, depending on factors such as, for example, the size of the gene in question and its location on the chromosome relative to the centromere. For example, for genes which are small or which occupy a very small part of a chromosome, the number of SNPs available within and either side of the gene which can be considered as proximate and/or linked may be less than for a larger gene. Without wishing to be bound by theory, it is thought that recombination events between the chromosomes are more likely to occur the further one moves away from the gene—as such a SNP which occupies a locus proximate (or close to) a gene is less likely to recombine with the corresponding locus on the opposite chromosome and as such is more likely to be associated with or linked to that gene.

By way of an example, in the case of the breast cancer genes BRCA1 and BRCA 2, SNPs which may be considered proximate may be those which occupy loci within about 10 MB of either gene, preferably about 8 MB, more preferably about 5 MB and more preferably within about 2 MB of the gene. In one embodiment, the selected SNPs occupy loci within about 1 MB of either BRCA 1 or BRCA2.

In the case of genes associated with hypertrophic cardiomyopathy and/or dilated cardiomyopathy, SNPs which may be considered proximate may be those which occupy loci within about 15 MB of either gene, preferably about 12 MB, more preferably about 10 MB and more preferably within about 5 MB of the gene. In one embodiment, the selected SNPs occupy loci within about 2.5 MB of the relevant gene (see for example those SNPs listed in Table 8 below (see also Table 9 for gene key)).

The at least two subjects are affected by the genetic disorder. By “affected”, it is meant that, in addition to having a suitable relationship as described above, the subjects are known to be suffering from or exhibiting symptoms of the genetic disorder. It is important to understand that it is not necessary to know whether or not each of the affected subjects harbours or carries the gene (i.e. the mutated or altered gene) that the method is seeking to eliminate as the likely cause of the genetic disorder in that family.

The term nucleic acid may be taken to include both deoxyribonucleic acid (DNA) and ribonucleic acid (RNA). In the latter case RNA may be taken to include all forms of RNA and in particular messenger RNA (mRNA). Preferably, the sample of nucleic acid is a sample of DNA.

Nucleic acid, for example DNA, may be obtained from almost any tissue or cell. For example, it may be possible to obtain a nucleic acid sample from a sample of tissue or from a biopsy. Additionally, or alternatively, samples of skin and/or samples of cells derived from the buccal cavity of a subject may be used. Such samples may be obtained by means of a swab or other sampling device. Alternatively samples of hair may be removed from the subject in a manner which ensures that at least a portion of the hair follicles and/or skin surrounding the hair is also removed.

In instances where an affected subject has deceased, it may still be possible to obtain nucleic acid, and particularly DNA, from samples which have been preserved. For example, it may be possible to extract nucleic acid for use in the methods described herein, from samples of tissue which have been preserved by methods involving paraffin embedding of the tissue.

The sample obtained may be subjected to a nucleic acid extracting protocol. Such protocols are well established and known to the skilled person (see for example Molecular Cloning: A Laboratory Manual (Third Edition); Sambrook et al.; CSHL Press). In addition a number of kits are available which facilitate the extraction of nucleic acid from a variety of sample types. Such kits are available from manufacturers such as, for example, Qiagen, Invitrogen LifeSciences and Amersham.

There a number of techniques which may be used to detect the selected SNPs in the nucleic acid samples provided by the at least two affected subjects linked by pedigree—many of which are known to one of skill in the art. For example, a number of polymerase chain reaction (PCR) based techniques may be used.

In one embodiment, oligonucleotide primers capable of hybridising to specific nucleic acid sequences, may be used together with polymerase enzymes, such as Taq polyemerase and nucleotides (dNTPs), to amplify particular regions of the nucleic acid. Preferably, the oligonucleotide primers may bind to regions which lie upstream and downstream of a particular SNP locus. Advantageously, the nucleic acid sequences amplified by PCR may be sequenced and aligned with a reference sequence such that regions and/or nucleotides which vary from the reference sequence may be easily identified. It is to be understood that the term “reference sequence” refers to a sequence or sequences obtained from one or more healthy individuals. In this way the presence of SNP alleles which are oppositely homozygous in at least two of the affected subjects may be identified.

In a further embodiment, a SNP allele detection technique or system such as the Illumina® Golden gate assay™ may be used to detect the presence of the selected SNPs in the nucleic acid samples. Briefly, oligonucleotide primers capable of hybridising to a region of nucleic acid comprising a target SNP (i.e. one of the SNPs selected in step (a) of the first aspect of this invention) may be used to detect the presence of particular SNP alleles in the nucleic acid sample. Oligonucleotide primers of this sort will be referred to hereinafter as “allele specific oligonucleotides” (ASO). Advantageously, a single ASO may be designed for each allele comprising the SNP. As such, if the SNP comprises two alleles, two ASOs should be designed—one for each allele.

Preferably, the ASOs may bind directly adjacent the target SNP. Advantageously, the ASO may comprise a 3′ nucleotide/base (preferably the most 3′ base) specific or complementary for/to one of the alleles which comprise the SNP. One of skill in the art will note that in instances where the target SNP comprises two alleles, two ASOs, each with a 3′ base specific for the alleles which comprise the SNP, will be required. In addition, the portion of the ASO immediately adjacent said 3′ base or nucleotide may be complementary to the sequence immediately upstream of the SNP allele. At the 5′ end, the ASO may comprise a sequence which is not complementary to the a nucleic acid sequence of the nucleic acid sample but which may comprise a sequence which is itself capable of binding to, or hybridising with, an oligonucleotide sequence.

Advantageously, one ASO specific for a particular SNP allele may comprise a sequence capable of binding one further nucleotide sequence and the ASO specific for the other SNP allele may bind a different further nucleic acid sequence. In one embodiment the further sequence may comprise a sequence capable of binding to or hybridising with a primer

In addition, the SNP allele detection technique suitable for use in the methods described herein may require the use of further oligonucleotide primers which are capable of binding or hybridising to a nucleic acid sequence which is located down stream of the target SNP. Oligonucleotide primers of this type will be referred to hereinafter as “locus specific oligonucleotides” (LSO). Advantageously, the LSO may bind to a nucleic acid sequence located downstream and on the same strand as, the ASO binding (or hybridisation) site. Preferably the LSO binds approximately 50 bases, more preferably 40 bases and even more preferably 30 bases downstream of the target SNP. In one embodiment, the LSO binds approximately 20 bases downstream of the target SNP. One of skill in the art will understand that the precise downstream distance is not crucial and indeed there is a degree of flexibility associated with the positioning of the LSO hybridisation site relative to the target SNP. In this way, when designing an assay capable of detecting more than one SNP in a nucleic acid sequence, it is possible to adjust the downstream position of the LSO relative to the target SNP such that the LSO does not hybridise at the site of another target SNP.

In one embodiment the LSO may comprise a nucleic acid sequence capable of binding additional nucleic acid sequences. For example, the LSO may comprise a sequence which itself is capable of hybridising to an oligonucleotide primer and/or an oligonucleotide probe or sequence.

Each of the ASOs specific for each of the alleles of the selected SNPs and the corresponding LSO may be contacted with the nucleic acid sample provided by each of the at least two affected individuals linked by pedigree, under conditions which permit binding of the oligonucleotides to their respective target sequences. One of skill will appreciate that only the ASO comprising the 3′ base specific for one of the alleles comprising the SNP will bind to the nucleic acid sequence.

Upon completion of the hybridisation step, the ASO may be extended with the use of a polymerase enzyme. Preferably, a polymerase enzyme, such as Taq polymerase, with a high specificity for 3′ mismatch may be used such that only those ASOs having a 3′ base which matches the SNP allele are extended.

After extension of the various bound ASOs is complete, the extended ASO sequence may be ligated to the LSO oligonucleotide by using, for example, a ligating compound such as, for example, a ligase enzyme.

The ligated extended ASO and LSO sequence will be referred to hereinafter as the “template sequence”. As stated above, the ASO and LSO sequences may comprise nucleic acid sequences capable of binding further nucleic acid sequences such as, for example, oligonucleotide primers (referred to hereinafter as “secondary primers”). As such, and in a further embodiment, the template sequence may be contacted with secondary primers which hybridise to nucleic acid sequences comprised within the LSO and ASO sequences of the template sequence. In one embodiment, the secondary primers capable of hybridising to a sequence of the ASOs may further comprise a detectable moiety. The term “detectable moiety” will be understood by those skilled in the art to encompass, for example fluorescent and/or radiolabelled compounds. More specifically, the detectable moiety may be a fluorophore compound such as CY3 and/or CY5.

Advantageously, the secondary primers which bind an ASO specific for one SNP allele may comprise a detectable moiety which is different from that of the secondary primer which binds an ASO specific for another allele of the same SNP. For example, one secondary primer may comprise the fluorophore compound CY3 and the other may comprise CY5. In this way it is possible to distinguish nucleic acid sequences comprising one SNP allele from those comprising another SNP allele.

In one embodiment, the secondary primers which hybridise to a sequence of the LSO are not labelled with a detectable moiety.

Preferably, the template sequence/secondary primer complex is subjected to a further amplification protocol so as to extend the sequence of the secondary primer bound or hybridised to the ASO towards the secondary primer bound to the LSO. As stated above the extended sequence may be ligated to the primer bound to the LSO sequence by means of a ligase enzyme.

The above described method will result in a nucleic acid sequence comprising a detectable moiety (referred to hereinafter as a “labelled sequence”) indicative of the particular SNP allele present in the nucleic acid of the at least two subjects affected by the genetic disorder and linked by pedigree.

In order to identify the SNP alleles present in a particular nucleic acid sample, the various labelled sequences resulting from the Goldengate® assay may be detected by exploiting sequences present in the labelled sequence. For example, the labelled sequence may comprise a nucleotide sequence capable of hybridising to an immobilised moiety. In one embodiment, the immobilised moiety may comprise a bead conjugated or otherwise bound to or associated with a nucleic acid sequence capable of hybridising to a sequence present in the labelled sequence generated by the Goldengate® Assay.

Systems such as the, Universal IllumiCode™ Array and/or Sentrix Array Matrix (Illumina) may be useful in the detection process and further information may be obtained from Gunderson et al., “Decoding randomly ordered DNA arrays”. Genome Research, May 2004).

A further method for use in identifying the SNPs present in the nucleic acid samples provided by each of the at least two affected subjects, may comprise the use of ASO primers (as described above) bound- or otherwise immobilised on or to, a support substrate such as, for example, those solid supports used to produce microarray chips. Suitable materials may include glass, plastic, nitrocellulose, agarose or the like. Preferably, the ASO primers may be arranged as an array such that each ASO specific for a particular SNP allele occupies a particular site or portion on a chip.

Advantageously, the LSOs described herein may, when used in this method, be labelled with any of the detectable moieties described above.

In order to detect the SNP alleles present in a nucleic acid sample, the nucleic acid may be contacted with the bound or immobilised ASO primers as described above and subjected to an amplification protocol such that only the ASO comprising the 3′ base specific for one of the alleles comprising the SNP will be extended. Advantageously, the extended ASO may be ligated to the labelled LSO.

Preferably, the method comprises the further step of denaturing and/or washing the genomic DNA/ASO::LSO complexes. In this way Genomic DNA which has bound to an ASO comprising a 3′ nucleotide not specific for a SNP allele present in the genomic sample is removed. Furthermore, a wash and/or denaturing step will leave only the extended ASO bound to the support substrate. Thereafter, one of skill in the art will appreciate that if the LSO comprises a detectable moiety, by detecting the presence of labelled LSO, it may be possible to determine the particular SNP allele present at a particular locus.

While the above described techniques and methods represent exemplary means of detecting SNP alleles present in nucleic acid samples, it is important to understand that almost any technique or technology which allows the analysis of large numbers of SNPs at once may be equally useful. For example, techniques such as RFLP analysis, MALDI-TOF or the SNPlex™ genotyping analysis system which enables the simultaneous genotyping of a number of SNPs, may be used. In addition, one of skill in this field will understand that techniques which exploit microsatellite markers may also be useful.

Any of the above-described SNP allele identification protocols may allow one of skill in the art to determine which SNPs are present in a nucleic acid sample and the particular SNP allele. In order to be able to exclude the gene as being involved in a particular genetic disorder (or causative of, or associated with, a particular genetic disorder), the at least two subjects must be shown to harbour oppositely homozygous SNP alleles at a given SNP locus. When referring to SNP alleles, the term “homozygous” is intended to encompass subjects who, at any given SNP locus, harbour the same SNP allele on each chromosome. As such, the term “oppositely homozygous” refers to at least two subjects who, at the same SNP locus, are homozygous for different SNP alleles.

In a second aspect, the present invention provides a method of determining whether or not a subject should be tested for the presence of mutations in a gene associated with or causative of, a genetic disorder, said method comprising the steps of:
(a) selecting a population of single nucleotide polymorphisms (SNPs) that
   i. are proximate to the gene; and
   ii. have a minor allele frequency sufficient to establish an appropriate level of heterozygosity; and
(b) identifying the SNPs in nucleic acid samples provided by each of at least two subjects affected by the genetic disorder and linked by pedigree to each other and said subject;

wherein, the presence of SNP alleles which are oppositely homozygous in at least two of said affected subjects, indicates that the subject need not be tested for mutations in the gene causative of, or associated with, the genetic disorder.

As stated above, to minimise the generation of uninformative data, the pedigree links that exist between the at least two affected subjects should not represent, constitute or comprise a parent/child link. Advantageously, suitable relationships between affected subjects linked by pedigree may include, for example sibling, cousin and aunt/uncle/niece/nephew relationships.

It is to be understood that if no oppositely homozygous SNP alleles are detected in at least two of the affected subjects, the subject may be given the option of further testing. The term “tested” as used herein may be taken to encompass the practice comprising the steps of the subject providing a nucleic acid sample to be sequenced and/or otherwise analysed for the presence of a mutation within a particular gene, associated with or causative of the genetic disorder. One of skill in the art will appreciate that by excluding a particular gene as associated with or causative of a particular genetic disorder, it may be possible to perform the appropriate testing or administer appropriate treatment more rapidly.

In one embodiment, the genetic disorder is a dominant genetic disorder such as familial breast cancer, hypertrophic cardiomyopathy or dilated cardiomyopathy. As such, the method according to the second aspect (and others described herein) may be used to determine whether or not a subject should be tested for the presence of a gene associated with or causative of, familial breast cancer, hypertrophic cardiomyopathy or dilated cardiomyopathy.

Preferably, the SNPs may be identified by any of the methods described herein and an exemplary method may be the Illumina® Golden gate assay™ substantially described above. Additionally or alternatively other techniques which permit the analysis of large numbers of SNPs at once may also be used including, for example, RFLP analysis, MALDI-TOF and/or analysis technology such as SNPlex™. Other useful techniques may include those which exploit microsatellite markers.

In a third aspect, there is provided a kit for use in any of the methods described herein, said kit comprising:

(a) oligonucleotide primers capable of hybridising upstream and down stream of nucleotide sequences comprising SNPs that:
(i) are proximate to the gene; and
(ii) have a minor allele frequency sufficient to establish an appropriate level of heterozygosity.

The terms “upstream” and “downstream” are well known to one of skill in the art and should be taken to mean 5′ and 3′ of a particular nucleotide sequence. Preferably, a pair of primers may be capable of hybridizing upstream and downstream (i.e. 5′ and 3′) of a nucleotide sequence which comprises a single SNP. Advantageously, the oligonucleotide primer which binds upstream (or 5′) of the SNP may bind immediately adjacent the SNP and comprise a 3′ nucleotide specific for a particular allele of that SNP.

Advantageously, the kit may comprise a polymerase enzyme capable of extending the oligonucleotide primer bound downstream of the SNP in the direction of, or towards the oligonucleotide primer bound upstream of the SNP. Preferably, a polymerase enzyme with a high specificity for 3′ mismatch may be used such that only those oligonucleotides having a 3′ base which matches the SNP allele are extended.

In one embodiment, the kit comprises oligonucleotides capable of hybridising upstream and down stream of nucleotide sequences comprising the SNPs identified in Table 3 and/or Table 8.

In a third aspect, the present invention provides data set comprising information pertaining to SNPs that:

(i) are proximate to a gene causative of or associated with a genetic disorder; and

(ii) have a minor allele frequency sufficient to establish an appropriate level of heterozygosity.

Advantageously, the data set may be stored in an electronic form and in a fourth aspect, there is provided a computer pre-loaded with the abovementioned data set. In one embodiment, the data set comprises the information contained in Table 3—i.e. information pertaining to SNPs which:

(i) are proximate to a the BRCA1 and/or BRCA2 gene causative of or associated with breast cancer; and

(ii) have a minor allele frequency sufficient to establish an appropriate level of heterozygosity.

In a further embodiment, the data set comprises the information contained in Table 8—i.e. information pertaining to SNPs which:

(i) are proximate to genes causative of or associated with hypertrophic cardiomyopathy or dilated cardiomyopathy (see Table 9 for gene number key); and

(ii) have a minor allele frequency sufficient to establish an appropriate level of heterozygosity.

### DETAILED DESCRIPTION

The present invention will now be described in more detail and with reference to the following figures which show

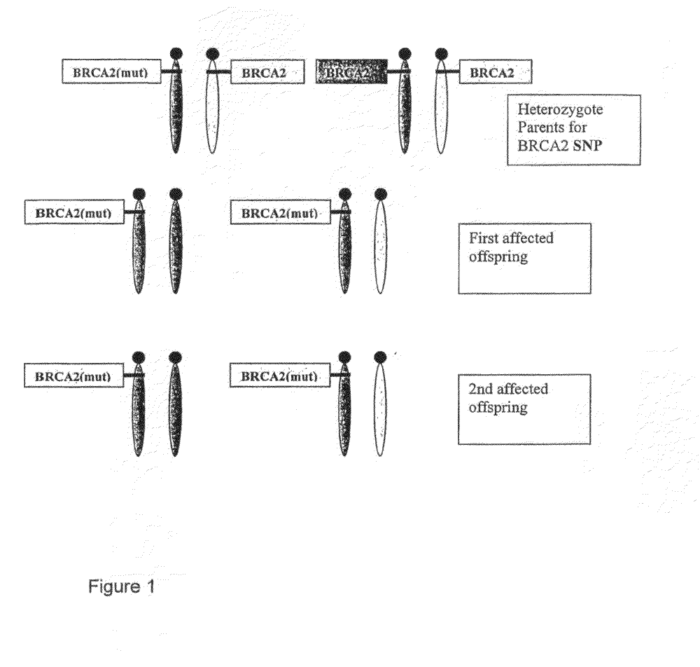

FIG. 1: Disease associated with BRCA2 will result in non-random segregation. There are only two possible segregations and both will share the chromosome carrying the BRCA2 mutation. If two affected relatives have breast cancer because they have inherited an altered BRCA gene they will both have inherited the gene from a common affected ancestor. In both individuals all the SNPs within and close to the abnormal gene will be inherited with the disease. The two affected individuals will therefore share these SNP alleles.

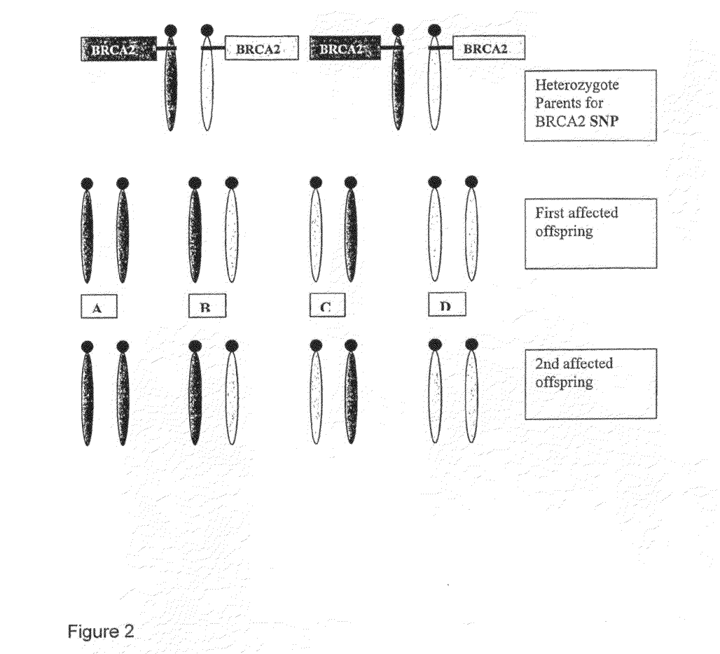

FIG. 2: Disease not associated with BRCA2 will result in random segregation in the offspring in keeping with Mendel's Laws. Affected A and D are oppositely homozygote and cannot share a copy of the BRCA 2 gene excluding this as the cause of the breast cancer in the family.

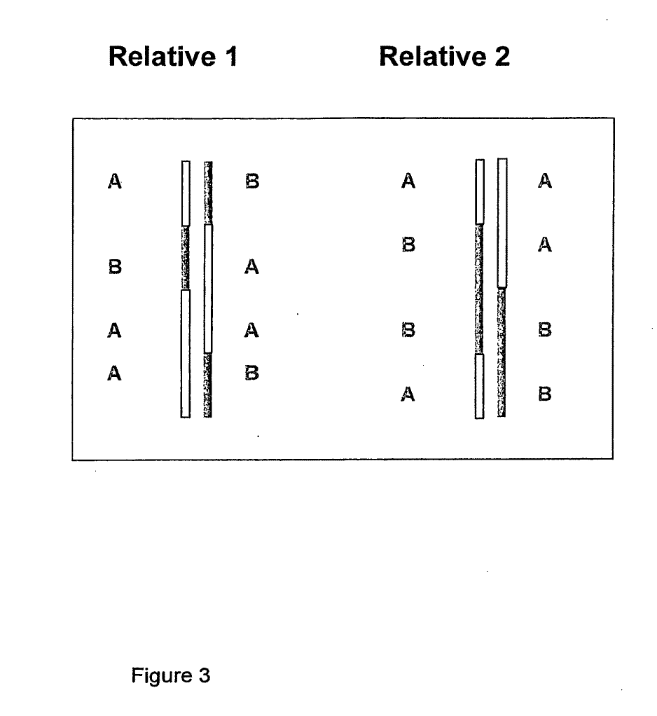

FIG. 3: Using SNPs to disprove linkage. If these were two breast cancer sufferers (Relative 1 and Relative 2) in a pedigree and the two diagrams represented their genotypes for 4 SNPs in the BRCA1 gene, being homozygous for different alleles in the 3rd SNP would suggest there was no linkage between the disease and the gene.

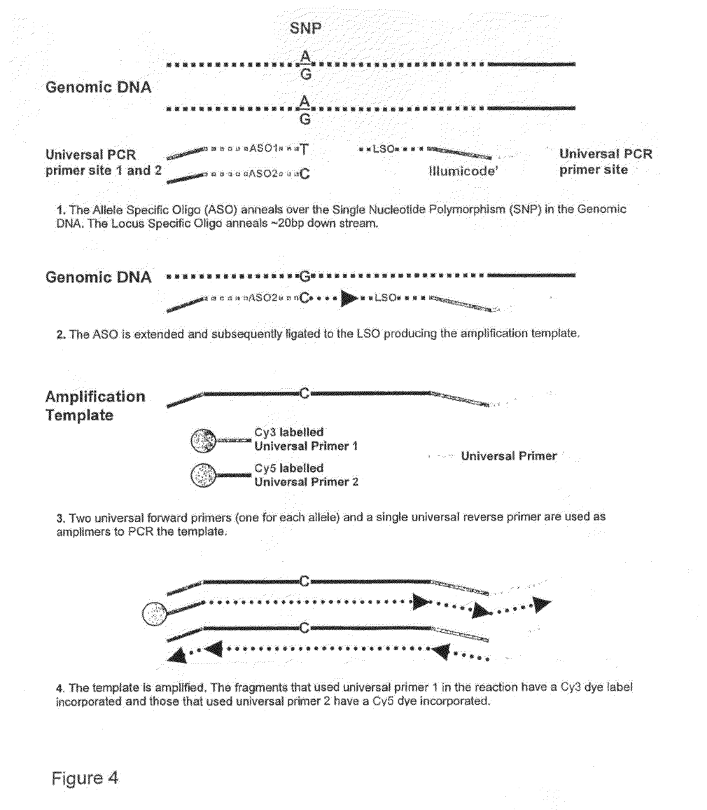

FIG. 4: Shows how a portion of genomic DNA comprising a SNP having two alleles may be subjected to the SNP allele detection, methods described herein. For each locus being interrogated 3 specific oligos are used, two allele specific oligos (ASOs), both of which have a sequence region that is a perfect complement to the genomic region directly adjacent to the target SNP site but that differ in their 3 prime base such that they only match one of the two alleles at the site, and a second region that acts as a universal primer site for the subsequent amplification reaction. A third locus specific oligo (LSO) hybridizes between 1 to 20 bases downstream of the target SNP site through a complementary sequence. The LSO also contains two other components, a unique sequence that perfectly matches an oligo on an array bead and a third universal primer sequence. After oligo hybridization, a polymerase with high specificity for 3′ mismatch is added and only extends the ASO(s) that perfectly match the target sequence at the SNP site. As the polymerase used has no strand displacement or exonuclease activity when it hits the LSO it simply drops off the genomic DNA. At this time a DNA Ligase joins the extended sequence to the LSO to from a superstructure that is a template for highly multiplexed PCR. After the high specificity extension and ligation reaction any ASO that matches a SNP will be incorporated into a super structure that is a perfect substrate for universal amplification. Amplification for all loci is completed with the addition of only 3 more primers. One universal primer labeled with Cy3 that hybridizes to Universal PCR sequence 1. Another universal primer labeled with Cy5 that hybridizes to Universal PCR sequence 2 and a third unlabeled primer for PCR sequence 3. Again only those ASOs that match the SNP and were extended from the super-structure and are amplified —confirming the alleles present at all sites.

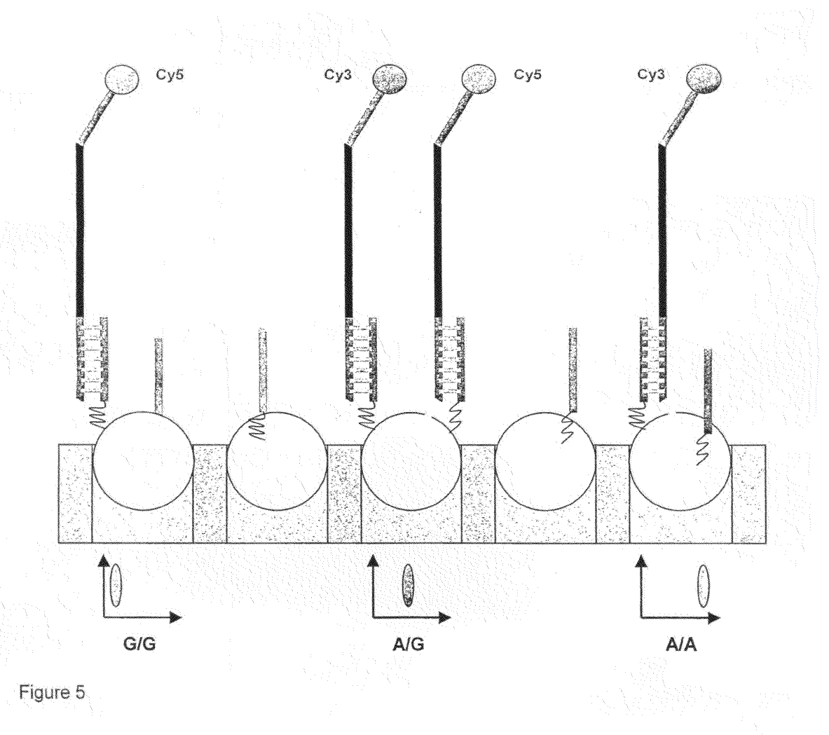

FIG. 5: After amplification the products are hybridized to the Sentrix array for detection. The internal code that is specific for each locus binds only to its complementary bead (the position of which was previously identified by decoding). The genotype is then easily and automatically called as loci that are homozygous for an allele will show signal in either the correlated green (Cy3) or red (Cy5) channel and those that are heterozygous show signal in both channels.

### MATERIALS AND METHODS

While the following methodology largely concerns the BRCA1 and BRCA2 genes, it is to be understood that the same experiments were conducted in relation to those genes known to be associated with or causative of hypertrophic cardiomyopathy or dilated cardiomyopathy.

DNA samples from the storage bank at the West of Scotland Regional Molecular Genetics Department were used for the study. Only samples from families that were known to have pathogenic gene mutations for BRCA1 or BRCA2 were chosen for the project. All samples that were chosen were linked by pedigree to at least one other sample; however, none were parent/child pairs. Similarly, DNA samples were obtained from families known to be affected by hypertrophic and dilated cardiomyopathy.

### SNP Selection

For the test to be informative for sibling pairs the parents must be heterozygous for the SNP. The likelihood of the parents being heterozygote will depend upon the gene frequency of the alleles of the SNP and becomes more likely as the allele frequencies approach 0.5. Furthermore, in order to maximise the chances of the test yielding informative data it is important to use enough SNP's.

The chance both parents are heterozygote is 2pq×2pq=4p²q² (Derived from the Hardy Weinberg):

$$Chance that a S N P may be informative = \frac{4 {p}^{2} {q}^{2}}{8} = \frac{{p}^{2} {q}^{2}}{2}$$

If p=0.3 and q=0.7
Chance informative p=0.022
If p=0.5 and q=0.5
Chance informative p=0.031

By selecting single nucleotide polymorphisms (SNPs) with appropriate levels of heterozygosity we can maximise the chance of finding two related individuals to be ‘oppositely homozygote’. This means that they do not share an allele at the locus being examined and the result will definitively exclude that locus.

Using the binomial distribution if 100 SNP's are selected per gene with allele frequencies between 0.3 and 0.5 the chance of observing at least one informative SNP is between 90 and 96%. If 150 SNPs are used the chance of at least one informative SNP will be between 96 and 99%. Using an oligo SNP array allows simultaneous testing of large numbers of SNPs and represents an entirely novel use of this technology.

For the BRCA1 and BRCA2 Breast Cancer study, single nucleotide polymorphisms (SNPs) were chosen based on their Minor Allele Frequency (MAF) and their proximity to the genes, BRCA 1 and 2. Only SNPs with a MAF of >0.3 and within 1 MB of the gene were deemed suitable due to the nature of the project. In all, 214 SNPs were chosen in and around BRCA1 and 170 in and around BRCA2. The aim of the SNP selection was to maximize the chances of finding the two patients homozygous for opposite alleles thereby excluding linkage in that area.

In the case of those genes known to be associated with or causative of hypertrophic or dilated cardiomyopathy, single nucleotide polymorphisms (SNPs) were also chosen on the basis of their MAF frequency and proximity to the gene. The table below (Table 1) shows the MAF value (i.e. the lowest MAF value any given SNP must possess to be considered for use in this study) for each of the genes selected.

TABLE 1
The Minor allele frequency of the SNPs used in the HOCM study

| Gene (associated with or causative | |
|---|---|
| of hypertrophic cardiomyopathy or | MAF of SNPs used |
| dilated cardiomyopathy) | in HOCM study |
| TTN | 0.1 |
| MYH6 and MYH7 | 0.13/0.2 |
| MYBPC3 | 0.2 |
| RAF1 | 0.1 |
| PRKAG2 | 0.225 |
| TPM1 | 0.3 |
| TNNT2 | 0.3 |
| MYLK2 | 0.14 |
| TNNI3 | 0.2 |
| MYL3 | 0.14 |
| MYL2 | 0.1 |
| CAV3 | 0.3 |

Based on the SNPs which we chose allele specific oligos (ASO) and locus specific oligos (LSO) were designed and manufactured by Illumina for use in the their ‘Golden Gate Assay’. The ASO hybridised specifically to a particular allele of the SNP and the LSO hybridised ˜20 bases downstream. Two ASOs were designed for each SNP locus, one for each allele. The LSO also contains a unique address sequence that targets bead types on the Sentrix Array Matrix (SAM). Both sets of oligonucleotides also contain universal primer binding sequences, 5′ in the ASOs and 3′ in the LSO. The Universal primers are denoted as P1 and P2, which are specific for the two different alleles and carry a Cy3- and Cy5-dyes respectively. P3 anneals 3′ of the LSO. Using these oligos Illumina manufactured the array to our specifications. All of the reagents except for the Taq polymerase were provided by Illumina and their analysis software was used to analyze the results from the array reader.

The Illumina Golden Gate Assay was carried out over 3 days according to the manufacturer's instructions. 250 ng of patient DNA samples were added to a 96 well micro titre plate. This DNA was subsequently activated for use with paramagnetic particles. The activated DNA was then combined with specific oligonucleotides, hybridisation buffer and paramagnetic particles in the hybridisation step. Several wash steps were performed post hybridisation to remove excess and mis-hybridised primers. The extension and ligation step seals the information regarding the allele present on the ASO and the specific address sequence on the LSO and provides a template for amplification using universal PCR amplicons. The samples were then amplified using the universal primers that annealed to a consensus sequence attached to each ASO and LSO. The final hybridisation used a Sentrix Array Matrix (SAM) that is composed of 96 fibre-optic bundles. The SAM is placed directly in to a micro titre plate carrying the PCR samples for direct hybridisation

The arrays were subsequently read using Illumina's plate reader and BeadScan software and the results analysed using Illumina BeadStudio software. The analysed results were then exported into a Microsoft Excel spreadsheet where sample genotypes were compared within their pedigrees

### Results

In all, 38 pedigrees were analysed, 27 of which had two relatives, 7 had three relatives and 4 had four relatives. In the families with two relatives, one comparison of alleles was made. With those with three relatives, three comparisons were made and those with four, six comparisons were made.

There were 52 sib pair comparisons, 23 were informative (44%), 9 aunt/uncle niece comparisons, 4 were informative (44%) and 16 cousin comparisons of which 13 were informative (81%)

In all 72 allelic comparisons were able to be made. 32 of these comparisons showed exclusion (LE) of one of the genes, 2 showed exclusion of both of the genes, 36 showed no evidence of exclusion and 2 failed.

Of the 34 that showed exclusion, 16 were BRCA1 (41% of BRCA1 comparisons) and 16 were BRCA2 (57% of BRCA2 comparisons).

From the 38 Pedigrees, 18 were BRCA2, of which 9 (50%) showed exclusion. The remaining 20 were BRCA1 pedigrees, of which 9 (45%) showed exclusion. The results are tabulated below (Table 2):

| | Total | Ped. Showing | | Total | Comp. showing | |
|---|---|---|---|---|---|---|
| | Pedigrees | exclusion | | Comparisons | exclusion | |
| BRCA1 | 20 | 7 | (19%) | 44 | 16 | (22%) |
| BRCA2 | 18 | 9 | (24%) | 28 | 16 | (22%) |
| Both | — | 2 | (5%) | — | 2 | (3%) |
| None | — | 18 | (47%) | — | 36 | (50%) |
| Failed | — | 2 | (5%) | — | 2 | (3%) |
| Total | 38 | 38 | | 72 | 72 | |

The above detailed results demonstrate that this method can be used to exclude the involvement of a locus using only two family members with a suitable relationship. The choice of SNPs is important in order to maximise the chances of finding the relatives oppositely homozygote and this is something which can be improved to reduce the number of uninformative samples. The use of opposite homozygotes as the criterion for the exclusion of a gene eliminates the need to establish phase and hence removes the requirement for many family members and a suitable family structure. This innovative idea makes the technique widely applicable in a diagnostic or a research setting to focus analysis on one relevant gene among several candidate genes.

We have already demonstrated its use to eliminate either BRCA 1 or 2 from analysis and the method may also be used to exclude both of these loci in families which do not have a mutation in either gene, potentially reducing the need to sequence by up to 70 or 80%. The object of finding a mutation in these breast cancer families is to provide predictive testing for other family members who may be at risk. Currently these concerned, at risk individuals have an anxious and often extended wait while a result is sought in their affected relative. Using the methods described herein, it would be possible in many cases to exclude linkage even before the sequencing results are obtained and reduce the anxiety of these patients.

TABLE 3
The SNPs selected as being proximate to the BRCA 1 and 2 genes and
having a MAF value sufficient to establish heterozygosity.

| Genomic Location | Chromosome | Name | BRCA |
|---|---|---|---|
| 30842815 | 13 | rs4941829 | 2 |
| 30853581 | 13 | rs2872422 | 2 |
| 30859924 | 13 | rs9531598 | 2 |
| 30866590 | 13 | rs4943360 | 2 |
| 30879675 | 13 | rs1359634 | 2 |
| 30887435 | 13 | rs4941842 | 2 |
| 30894875 | 13 | rs7321575 | 2 |
| 30959209 | 13 | rs2245328 | 2 |
| 30963121 | 13 | rs492821 | 2 |
| 30970018 | 13 | rs555163 | 2 |
| 30972743 | 13 | rs1854427 | 2 |
| 30976343 | 13 | rs660687 | 2 |
| 30985599 | 13 | rs277151 | 2 |
| 30985892 | 13 | rs277154 | 2 |
| 30995631 | 13 | rs277126 | 2 |
| 30999138 | 13 | rs535962 | 2 |
| 30999896 | 13 | rs598700 | 2 |
| 31003351 | 13 | rs1869799 | 2 |
| 31012398 | 13 | rs1535532 | 2 |
| 31026476 | 13 | rs1410810 | 2 |
| 31030567 | 13 | rs277143 | 2 |
| 31036103 | 13 | rs1328945 | 2 |
| 31042160 | 13 | rs277187 | 2 |
| 31045296 | 13 | rs1972210 | 2 |
| 31064025 | 13 | rs7328697 | 2 |
| 31078136 | 13 | rs1869800 | 2 |
| 31085369 | 13 | rs1671967 | 2 |
| 31087143 | 13 | rs1006096 | 2 |
| 31091225 | 13 | rs4943595 | 2 |
| 31103642 | 13 | rs1200453 | 2 |
| 31123592 | 13 | rs6563643 | 2 |
| 31124399 | 13 | rs2025908 | 2 |
| 31219924 | 13 | rs9594369 | 2 |
| 31225217 | 13 | rs1885637 | 2 |
| 31234040 | 13 | rs1536635 | 2 |
| 31246573 | 13 | rs7333829 | 2 |
| 31253432 | 13 | rs1555633 | 2 |
| 31258349 | 13 | rs1322575 | 2 |
| 31263271 | 13 | rs9594399 | 2 |
| 31268660 | 13 | rs7987979 | 2 |
| 31272281 | 13 | rs2038729 | 2 |
| 31284994 | 13 | rs2872643 | 2 |
| 31288588 | 13 | rs7139573 | 2 |
| 31295113 | 13 | rs9532560 | 2 |
| 31305576 | 13 | rs1575345 | 2 |
| 31311678 | 13 | rs1407521 | 2 |
| 31319604 | 13 | rs635865 | 2 |
| 31327355 | 13 | rs524566 | 2 |
| 31331799 | 13 | rs594468 | 2 |
| 31337508 | 13 | rs643595 | 2 |
| 31343743 | 13 | rs668574 | 2 |
| 31351583 | 13 | rs672839 | 2 |
| 31356802 | 13 | rs357 | 2 |
| 31365899 | 13 | rs203424 | 2 |
| 31366990 | 13 | rs23506 | 2 |
| 31370273 | 13 | rs619396 | 2 |
| 31377357 | 13 | rs366 | 2 |
| 31381206 | 13 | rs582274 | 2 |
| 31387796 | 13 | rs203415 | 2 |
| 31393061 | 13 | rs203432 | 2 |
| 31399506 | 13 | rs381 | 2 |
| 31504069 | 13 | rs424118 | 2 |
| 31506748 | 13 | rs204127 | 2 |
| 31512966 | 13 | rs369666 | 2 |
| 31525847 | 13 | rs392455 | 2 |
| 31527803 | 13 | rs398251 | 2 |
| 31536866 | 13 | rs390704 | 2 |
| 31552194 | 13 | rs204568 | 2 |
| 31559712 | 13 | rs204566 | 2 |
| 31574114 | 13 | rs364995 | 2 |
| 31581100 | 13 | rs387139 | 2 |
| 31581609 | 13 | rs396150 | 2 |
| 31588975 | 13 | rs176059 | 2 |
| 31595787 | 13 | rs799054 | 2 |
| 31645122 | 13 | rs916732 | 2 |
| 31646881 | 13 | rs798979 | 2 |
| 31648910 | 13 | rs703220 | 2 |
| 31652278 | 13 | rs2520688 | 2 |
| 31664817 | 13 | rs798961 | 2 |
| 31669704 | 13 | rs2188584 | 2 |
| 31671359 | 13 | rs881917 | 2 |
| 31671559 | 13 | rs2520696 | 2 |
| 31676223 | 13 | rs715129 | 2 |
| 31679313 | 13 | rs798990 | 2 |
| 31682082 | 13 | rs798993 | 2 |
| 31682594 | 13 | rs798994 | 2 |
| 31685876 | 13 | rs798987 | 2 |
| 31686225 | 13 | rs798988 | 2 |
| 31726553 | 13 | rs2157961 | 2 |
| 31730350 | 13 | rs2806636 | 2 |
| 31739619 | 13 | rs1380945 | 2 |
| 31743230 | 13 | rs2126043 | 2 |
| 31770987 | 13 | rs1126250 | 2 |
| 31777312 | 13 | rs206140 | 2 |
| 31782771 | 13 | rs206111 | 2 |
| 31786473 | 13 | rs206115 | 2 |
| 31818618 | 13 | rs206079 | 2 |
| 31834646 | 13 | rs9534262 | 2 |
| 31851388 | 13 | rs4942486 | 2 |
| 31857839 | 13 | rs2238163 | 2 |
| 31868736 | 13 | rs1012129 | 2 |
| 31875496 | 13 | rs1207952 | 2 |
| 31877647 | 13 | rs2761367 | 2 |
| 31884219 | 13 | rs206319 | 2 |
| 31912487 | 13 | rs2146994 | 2 |
| 31921729 | 13 | rs1207945 | 2 |
| 31928098 | 13 | rs1207948 | 2 |
| 31934660 | 13 | rs1088550 | 2 |
| 31940802 | 13 | rs703225 | 2 |
| 31958391 | 13 | rs207649 | 2 |
| 31971693 | 13 | rs798266 | 2 |
| 31981371 | 13 | rs207632 | 2 |
| 31993620 | 13 | rs621450 | 2 |
| 31997321 | 13 | rs535783 | 2 |
| 32051716 | 13 | rs208417 | 2 |
| 32052022 | 13 | rs208418 | 2 |
| 32057228 | 13 | rs129480 | 2 |
| 32089371 | 13 | rs208426 | 2 |
| 32104083 | 13 | rs208411 | 2 |
| 32117811 | 13 | rs9315173 | 2 |
| 32121444 | 13 | rs2301390 | 2 |
| 32125912 | 13 | rs12428283 | 2 |
| 32130435 | 13 | rs2301393 | 2 |
| 32135222 | 13 | rs4942791 | 2 |
| 32140535 | 13 | rs2301391 | 2 |
| 32154987 | 13 | rs687416 | 2 |
| 32156502 | 13 | rs4942804 | 2 |
| 32161714 | 13 | rs590383 | 2 |
| 32166573 | 13 | rs561056 | 2 |
| 32175086 | 13 | rs4120131 | 2 |
| 32179832 | 13 | rs1029306 | 2 |
| 32182645 | 13 | rs2183873 | 2 |
| 32187750 | 13 | rs9652200 | 2 |
| 32191268 | 13 | rs962454 | 2 |
| 32196533 | 13 | rs7322827 | 2 |
| 32205417 | 13 | rs7319275 | 2 |
| 32205684 | 13 | rs1929939 | 2 |
| 32213580 | 13 | rs762900 | 2 |
| 32219091 | 13 | rs2051570 | 2 |
| 32221486 | 13 | rs7318563 | 2 |
| 32226002 | 13 | rs7331632 | 2 |
| 32231368 | 13 | rs1924606 | 2 |
| 32236360 | 13 | rs9596218 | 2 |
| 32243103 | 13 | rs3752476 | 2 |
| 32247341 | 13 | rs3492 | 2 |
| 32253337 | 13 | rs1924605 | 2 |
| 32263770 | 13 | rs2025473 | 2 |
| 32274884 | 13 | rs8001047 | 2 |
| 32279025 | 13 | rs4942923 | 2 |
| 32485652 | 13 | rs398655 | 2 |
| 32494189 | 13 | rs211239 | 2 |
| 32528055 | 13 | rs522796 | 2 |
| 32542537 | 13 | rs582524 | 2 |
| 32548279 | 13 | rs537008 | 2 |
| 32583621 | 13 | rs540105 | 2 |
| 32594821 | 13 | rs487405 | 2 |
| 32601656 | 13 | rs495680 | 2 |
| 32604179 | 13 | rs626326 | 2 |
| 32607803 | 13 | rs653938 | 2 |
| 32613437 | 13 | rs2005641 | 2 |
| 32618790 | 13 | rs523523 | 2 |
| 32630440 | 13 | rs961106 | 2 |
| 32652516 | 13 | rs2555607 | 2 |
| 32684649 | 13 | rs2858130 | 2 |
| 32741642 | 13 | rs6561812 | 2 |
| 32748251 | 13 | rs3742323 | 2 |
| 32751684 | 13 | rs2321169 | 2 |
| 32753398 | 13 | rs1059180 | 2 |
| 32756313 | 13 | rs7983466 | 2 |
| 32794531 | 13 | rs3848097 | 2 |
| 37472959 | 17 | rs1076188 | 1 |
| 37480879 | 17 | rs7224322 | 1 |
| 37489698 | 17 | rs2023798 | 1 |
| 37525283 | 17 | rs730086 | 1 |
| 37680015 | 17 | rs7209222 | 1 |
| 37719618 | 17 | rs1053004 | 1 |
| 37746413 | 17 | rs3869550 | 1 |
| 37748916 | 17 | rs8072391 | 1 |
| 37749550 | 17 | rs7217655 | 1 |
| 37751361 | 17 | rs3736161 | 1 |
| 37751799 | 17 | rs3816769 | 1 |
| 37753059 | 17 | rs6503695 | 1 |
| 37753791 | 17 | rs2306581 | 1 |
| 37761506 | 17 | rs9891119 | 1 |
| 37767727 | 17 | rs744166 | 1 |
| 37773451 | 17 | rs957971 | 1 |
| 37779799 | 17 | rs12949918 | 1 |
| 37783361 | 17 | rs1026916 | 1 |
| 37784289 | 17 | rs4796791 | 1 |
| 37785287 | 17 | rs7219739 | 1 |
| 37787601 | 17 | rs7211777 | 1 |
| 37806320 | 17 | rs2883456 | 1 |
| 37815278 | 17 | rs963988 | 1 |
| 37824128 | 17 | rs12948909 | 1 |
| 37825773 | 17 | rs8065702 | 1 |
| 37826806 | 17 | rs11656652 | 1 |
| 37835622 | 17 | rs1905339 | 1 |
| 37864814 | 17 | rs2301647 | 1 |
| 37892948 | 17 | rs8069972 | 1 |
| 37952068 | 17 | rs12453490 | 1 |
| 37958089 | 17 | rs2830 | 1 |
| 37958626 | 17 | rs597255 | 1 |
| 37960970 | 17 | rs592389 | 1 |
| 37965295 | 17 | rs12602084 | 1 |
| 37967540 | 17 | rs629861 | 1 |
| 37969761 | 17 | rs668799 | 1 |
| 37970046 | 17 | rs598126 | 1 |
| 37974158 | 17 | rs646123 | 1 |
| 37975376 | 17 | rs17671179 | 1 |
| 37979182 | 17 | rs2292752 | 1 |
| 37981755 | 17 | rs878291 | 1 |
| 37985123 | 17 | rs6963 | 1 |
| 37988995 | 17 | rs1517002 | 1 |
| 37989167 | 17 | rs10454087 | 1 |
| 37990643 | 17 | rs4792992 | 1 |
| 38002263 | 17 | rs4793056 | 1 |
| 38011090 | 17 | rs4793036 | 1 |
| 38013655 | 17 | rs3760389 | 1 |
| 38021214 | 17 | rs9911799 | 1 |
| 38025923 | 17 | rs11649877 | 1 |
| 38030777 | 17 | rs2089114 | 1 |
| 38032294 | 17 | rs4277395 | 1 |
| 38059370 | 17 | rs4792930 | 1 |
| 38065307 | 17 | rs2292750 | 1 |
| 38070974 | 17 | rs3817331 | 1 |
| 38072225 | 17 | rs1046097 | 1 |
| 38089175 | 17 | rs4028634 | 1 |
| 38089448 | 17 | rs2271029 | 1 |
| 38094923 | 17 | rs2271028 | 1 |
| 38095610 | 17 | rs3760386 | 1 |
| 38103368 | 17 | rs1553468 | 1 |
| 38112367 | 17 | rs2089115 | 1 |
| 38114950 | 17 | rs2242461 | 1 |
| 38126829 | 17 | rs4792953 | 1 |
| 38149603 | 17 | rs7215553 | 1 |
| 38155350 | 17 | rs752313 | 1 |
| 38166286 | 17 | rs1078523 | 1 |
| 38257494 | 17 | rs630079 | 1 |
| 38324922 | 17 | rs692198 | 1 |
| 38328013 | 17 | rs323495 | 1 |
| 38329067 | 17 | rs323494 | 1 |
| 38357638 | 17 | rs2292539 | 1 |
| 38374789 | 17 | rs4793187 | 1 |
| 38380974 | 17 | rs6503721 | 1 |
| 38391027 | 17 | rs12944462 | 1 |
| 38407743 | 17 | rs1567832 | 1 |
| 38419260 | 17 | rs443759 | 1 |
| 38447436 | 17 | rs8071278 | 1 |
| 38448551 | 17 | rs11659028 | 1 |
| 38450800 | 17 | rs8176318 | 1 |
| 38456851 | 17 | rs8176297 | 1 |
| 38457117 | 17 | rs8176296 | 1 |
| 38469351 | 17 | rs3092994 | 1 |
| 38471859 | 17 | rs4793194 | 1 |
| 38476620 | 17 | rs1799966 | 1 |
| 38480201 | 17 | rs2236762 | 1 |
| 38485428 | 17 | rs4793197 | 1 |
| 38487996 | 17 | rs1060915 | 1 |
| 38489325 | 17 | rs8067269 | 1 |
| 38495029 | 17 | rs8176160 | 1 |
| 38495811 | 17 | rs2070834 | 1 |
| 38496716 | 17 | rs799916 | 1 |
| 38497526 | 17 | rs16942 | 1 |
| 38497961 | 17 | rs16941 | 1 |
| 38498462 | 17 | rs799917 | 1 |
| 38498763 | 17 | rs16940 | 1 |
| 38498992 | 17 | rs1799949 | 1 |
| 38505172 | 17 | rs8176140 | 1 |
| 38505457 | 17 | rs799923 | 1 |
| 38510660 | 17 | rs799912 | 1 |
| 38519302 | 17 | rs8176109 | 1 |
| 38520576 | 17 | rs8176103 | 1 |
| 38529773 | 17 | rs3765640 | 1 |
| 38530713 | 17 | rs799905 | 1 |
| 38531642 | 17 | rs799906 | 1 |
| 38540348 | 17 | rs2175957 | 1 |
| 38545479 | 17 | rs11657835 | 1 |
| 38559352 | 17 | rs2037075 | 1 |
| 38564021 | 17 | rs1973646 | 1 |
| 38581147 | 17 | rs2271573 | 1 |
| 38581621 | 17 | rs4239149 | 1 |
| 38584832 | 17 | rs11653460 | 1 |
| 38585342 | 17 | rs12936831 | 1 |
| 38595510 | 17 | rs8070085 | 1 |
| 38708936 | 17 | rs17599948 | 1 |
| 38727370 | 17 | rs13851 | 1 |
| 38731081 | 17 | rs17600371 | 1 |
| 38748821 | 17 | rs17600502 | 1 |
| 38773860 | 17 | rs4793229 | 1 |
| 38777402 | 17 | rs1824889 | 1 |
| 38779627 | 17 | rs9646416 | 1 |
| 38779779 | 17 | rs9646417 | 1 |
| 38780091 | 17 | rs1545764 | 1 |
| 38780445 | 17 | rs4584865 | 1 |
| 38782995 | 17 | rs4793231 | 1 |
| 38800671 | 17 | rs9912203 | 1 |
| 38818201 | 17 | rs880690 | 1 |
| 38821393 | 17 | rs12600401 | 1 |
| 38826209 | 17 | rs4793248 | 1 |
| 38845687 | 17 | rs8068764 | 1 |
| 38847298 | 17 | rs12601926 | 1 |
| 38850949 | 17 | rs11079338 | 1 |
| 38856821 | 17 | rs7215223 | 1 |
| 38866376 | 17 | rs11079387 | 1 |
| 38877192 | 17 | rs1984518 | 1 |
| 38882052 | 17 | rs732508 | 1 |
| 38901649 | 17 | rs1463558 | 1 |
| 38916260 | 17 | rs7214914 | 1 |
| 38958044 | 17 | rs1971 | 1 |
| 38985576 | 17 | rs1573938 | 1 |
| 38995083 | 17 | rs2343133 | 1 |
| 39000770 | 17 | rs17602795 | 1 |
| 39005538 | 17 | rs939357 | 1 |
| 39007602 | 17 | rs4792997 | 1 |
| 39008984 | 17 | rs7210301 | 1 |
| 39010566 | 17 | rs4352089 | 1 |
| 39015607 | 17 | rs4792901 | 1 |
| 39021949 | 17 | rs939358 | 1 |
| 39023053 | 17 | rs11655135 | 1 |
| 39029516 | 17 | rs1320304 | 1 |
| 39034239 | 17 | rs16940030 | 1 |
| 39034501 | 17 | rs11656202 | 1 |
| 39045531 | 17 | rs9916473 | 1 |
| 39045774 | 17 | rs4793007 | 1 |
| 39047052 | 17 | rs1004357 | 1 |
| 39055489 | 17 | rs575873 | 1 |
| 39067282 | 17 | rs658261 | 1 |
| 39069204 | 17 | rs479249 | 1 |
| 39070759 | 17 | rs8081000 | 1 |
| 39086873 | 17 | rs16940078 | 1 |
| 39089662 | 17 | rs1811196 | 1 |
| 39090646 | 17 | rs739769 | 1 |
| 39090873 | 17 | rs739770 | 1 |
| 39103977 | 17 | rs1398882 | 1 |
| 39109982 | 17 | rs8070993 | 1 |
| 39115918 | 17 | rs1398883 | 1 |
| 39117267 | 17 | rs11652705 | 1 |
| 39118689 | 17 | rs1694010 | 1 |
| 39124661 | 17 | rs9893304 | 1 |
| 39129340 | 17 | rs1107748 | 1 |
| 39130569 | 17 | rs1877633 | 1 |
| 39132812 | 17 | rs4793016 | 1 |
| 39136506 | 17 | rs4793017 | 1 |
| 39143047 | 17 | rs9909172 | 1 |
| 39145491 | 17 | rs7220711 | 1 |
| 39153218 | 17 | rs1828720 | 1 |
| 39154381 | 17 | rs4793022 | 1 |
| 39155116 | 17 | rs1877632 | 1 |
| 39159990 | 17 | rs6416905 | 1 |
| 39162857 | 17 | rs1513670 | 1 |
| 39165650 | 17 | rs1534401 | 1 |
| 39165839 | 17 | rs8071941 | 1 |
| 39168087 | 17 | rs955412 | 1 |
| 39170502 | 17 | rs9915878 | 1 |
| 39171292 | 17 | rs9303539 | 1 |
| 39180165 | 17 | rs7342964 | 1 |
| 39184822 | 17 | rs851062 | 1 |
| 39192149 | 17 | rs851054 | 1 |
| 39193245 | 17 | rs851058 | 1 |
| 39198260 | 17 | rs1230394 | 1 |
| 39198555 | 17 | rs1230396 | 1 |
| 39199505 | 17 | rs15359 | 1 |
| 39293608 | 17 | rs11867362 | 1 |
| 39339014 | 17 | rs231481 | 1 |
| 39339362 | 17 | rs1731903 | 1 |
| 39342588 | 17 | rs3744416 | 1 |
| 39342967 | 17 | rs105025 | 1 |
| 39344518 | 17 | rs3760360 | 1 |
| 39346388 | 17 | rs1631484 | 1 |
| 39347501 | 17 | rs1731889 | 1 |
| 39348247 | 17 | rs1731887 | 1 |
| 39348543 | 17 | rs1662746 | 1 |
| 39357548 | 17 | rs231452 | 1 |
| 39357972 | 17 | rs1731900 | 1 |
| 39365771 | 17 | rs1662748 | 1 |
| 39368452 | 17 | rs1731893 | 1 |
| 39375020 | 17 | rs231471 | 1 |
| 39377233 | 17 | rs1642603 | 1 |
| 39381051 | 17 | rs1662754 | 1 |
| 39389479 | 17 | rs419168 | 1 |
| 39410695 | 17 | rs1684668 | 1 |
| 39421451 | 17 | rs1618809 | 1 |
| 39441498 | 17 | rs186636 | 1 |
| 39446113 | 17 | rs228778 | 1 |

### Results—Hypertrophic Obstructive Cardiomyopathy Study (HOCM)

TABLE 4
Average number of genes excluded in different family pairs.

| | Number of | Average number |
|---|---|---|
| Relationship | pairs | genes excluded |
| Sibs | 49 | 3 |
| Aunt/Niece Nephew | 22 | 5 |
| First Cousins | 7 | 7 |
| First Cousins | 5 | 8 |
| once removed | | |
| Second Cousins | 9 | 7 |

TABLE 5
Average number of times each gene has been excluded
in the entire HOCM plate (96 pair comparisons)

| | Number of times | Percentage number of |
|---|---|---|
| Gene | gene excluded | times gene excluded |
| TTN | 34/96 | 35% |
| MYH6 and MYH7 | 33/96 | 34% |
| MYBPC3 | 28/96 | 29% |
| RAF1 | 23/96 | 24% |
| PRKAG2 | 47/96 | 49% |
| TPM1 | 29/96 | 30% |
| TNNT2 | 39/96 | 41% |
| MYLK2 | 40/96 | 42% |
| TNNI3 | 34/96 | 35% |
| MYL3 | 33/96 | 34% |
| MYL2 | 26/96 | 27% |
| CAV3 | 40/96 | 42% |

TABLE 6
20 families out of 28 have one known mutation.

| Location of known | No families with | % families with |
|---|---|---|
| mutation | known mutation | known mutation |
| MYH6 or 7 | 5/20 | 25% |
| MYBPC3 | 3/20 | 15% |
| PRKAG2 | 1/20 | 5% |
| TPM1 | 3/20 | 15% |
| TNNT2 | 4/20 | 20% |
| TNNI3 | 3/20 | 15% |
| MYL3 | 1/20 | 5% |

TABLE 7
Number of SNPs used for each gene, as well as the
actual number that clustered well upon analysis
and were able to be used for the comparisons.

| | | No SNPs actually | % SNPs used for |
|---|---|---|---|
| Gene | No SNPs | used for analysis | analysis |
| TTN | 212 | 190 | 90% |
| MYH6 and 7 | 153 | 134 | 88% |
| MYBPC3 | 147 | 130 | 88% |
| RAF1 | 163 | 138 | 85% |
| PRKAG2 | 146 | 121 | 83% |
| TPM1 | 132 | 120 | 91% |
| TNNT2 | 116 | 99 | 85% |
| MYLK2 | 94 | 82 | 87% |
| TNNI3 | 92 | 76 | 83% |
| MYL3 | 89 | 74 | 83% |
| MYL2 | 94 | 81 | 86% |
| CAV3 | 98 | 86 | 88% |

TABLE 8
The SNPs selected as being proximate to each of genes 1-12 associated
with or causative of hypertrophic cardiomyopathy and dilated
cardiomyopathy having a MAF value sufficient
to establish heterozygosity.

| Genomic Location | Chr | SNP name | Gene |
|---|---|---|---|
| 199345711 | 1 | rs1325311 | 1 |
| 199346081 | 1 | rs1325309 | 1 |
| 199352043 | 1 | rs12079819 | 1 |
| 199355512 | 1 | rs942702 | 1 |
| 199359602 | 1 | rs942703 | 1 |
| 199367105 | 1 | rs10753891 | 1 |
| 199368036 | 1 | rs6697303 | 1 |
| 199371212 | 1 | rs1059625 | 1 |
| 199376000 | 1 | rs10920128 | 1 |
| 199379941 | 1 | rs10920130 | 1 |
| 199380020 | 1 | rs7526291 | 1 |
| 199383534 | 1 | rs6664624 | 1 |
| 199387353 | 1 | rs2068152 | 1 |
| 199390422 | 1 | rs1106729 | 1 |
| 199391949 | 1 | rs7542768 | 1 |
| 199400397 | 1 | rs863826 | 1 |
| 199400578 | 1 | rs831748 | 1 |
| 199404123 | 1 | rs875495 | 1 |
| 199405051 | 1 | rs1122396 | 1 |
| 199405940 | 1 | rs1534052 | 1 |
| 199410397 | 1 | rs2066284 | 1 |
| 199412576 | 1 | rs831772 | 1 |
| 199415218 | 1 | rs4915492 | 1 |
| 199421670 | 1 | rs831757 | 1 |
| 199426084 | 1 | rs831750 | 1 |
| 199429814 | 1 | rs1107128 | 1 |
| 199430207 | 1 | rs1568809 | 1 |
| 199430862 | 1 | rs6690367 | 1 |
| 199431387 | 1 | rs17508253 | 1 |
| 199431866 | 1 | rs4915220 | 1 |
| 199432747 | 1 | rs12045948 | 1 |
| 199432904 | 1 | rs7533096 | 1 |
| 199433006 | 1 | rs4915221 | 1 |
| 199443591 | 1 | rs12063867 | 1 |
| 199447840 | 1 | rs12070918 | 1 |
| 199449402 | 1 | rs2282413 | 1 |
| 199449787 | 1 | rs2282414 | 1 |
| 199449986 | 1 | rs2282415 | 1 |
| 199461742 | 1 | rs3738270 | 1 |
| 199463617 | 1 | rs6692583 | 1 |
| 199463954 | 1 | rs3753971 | 1 |
| 199465761 | 1 | rs10920147 | 1 |
| 199483771 | 1 | rs10920155 | 1 |
| 199489605 | 1 | rs6427895 | 1 |
| 199502862 | 1 | rs10920161 | 1 |
| 199503300 | 1 | rs1997018 | 1 |
| 199504430 | 1 | rs1857489 | 1 |
| 199510674 | 1 | rs1568811 | 1 |
| 199512643 | 1 | rs12128083 | 1 |
| 199513259 | 1 | rs832179 | 1 |
| 199517939 | 1 | rs854488 | 1 |
| 199522140 | 1 | rs832165 | 1 |
| 199522315 | 1 | rs832166 | 1 |
| 199526268 | 1 | rs832175 | 1 |
| 199528807 | 1 | rs2268153 | 1 |
| 199532393 | 1 | rs713292 | 1 |
| 199533058 | 1 | rs1318899 | 1 |
| 199533697 | 1 | rs2268156 | 1 |
| 199542404 | 1 | rs1772836 | 1 |
| 199546348 | 1 | rs1779284 | 1 |
| 199547780 | 1 | rs832150 | 1 |
| 199548473 | 1 | rs1772858 | 1 |
| 199552941 | 1 | rs1722780 | 1 |
| 199553301 | 1 | rs1794867 | 1 |
| 199554152 | 1 | rs916398 | 1 |
| 199555138 | 1 | rs1794870 | 1 |
| 199556326 | 1 | rs1628556 | 1 |
| 199562657 | 1 | rs3767518 | 1 |
| 199562787 | 1 | rs3767519 | 1 |
| 199563110 | 1 | rs854508 | 1 |
| 199563301 | 1 | rs854509 | 1 |
| 199565489 | 1 | rs1361902 | 1 |
| 199598287 | 1 | rs2365652 | 1 |
| 199603264 | 1 | rs1892028 | 1 |
| 199611827 | 1 | rs947486 | 1 |
| 199622145 | 1 | rs4128458 | 1 |
| 199631193 | 1 | rs4606345 | 1 |
| 199639336 | 1 | rs2799681 | 1 |
| 199642281 | 1 | rs1256945 | 1 |
| 199642374 | 1 | rs1256946 | 1 |
| 199642630 | 1 | rs12141855 | 1 |
| 199642884 | 1 | rs1256948 | 1 |
| 199648284 | 1 | rs3850626 | 1 |
| 199651741 | 1 | rs12116834 | 1 |
| 199658686 | 1 | rs3738287 | 1 |
| 199664851 | 1 | rs2015284 | 1 |
| 199666484 | 1 | rs10920193 | 1 |
| 199666711 | 1 | rs10800777 | 1 |
| 199677208 | 1 | rs12408988 | 1 |
| 199685998 | 1 | rs7525711 | 1 |
| 199688824 | 1 | rs10920205 | 1 |
| 199690563 | 1 | rs3887566 | 1 |
| 199692408 | 1 | rs11586517 | 1 |
| 199696004 | 1 | rs12090950 | 1 |
| 199696908 | 1 | rs11591030 | 1 |
| 199701363 | 1 | rs1053592 | 1 |
| 199723422 | 1 | rs12729389 | 1 |
| 199723837 | 1 | rs12403361 | 1 |
| 199725145 | 1 | rs11585456 | 1 |
| 199726244 | 1 | rs927901 | 1 |
| 199726699 | 1 | rs3767538 | 1 |
| 199728432 | 1 | rs495886 | 1 |
| 199730063 | 1 | rs680198 | 1 |
| 199730548 | 1 | rs682137 | 1 |
| 199731430 | 1 | rs515384 | 1 |
| 199732567 | 1 | rs4915529 | 1 |
| 199739577 | 1 | rs592865 | 1 |
| 199744899 | 1 | rs3767525 | 1 |
| 199745752 | 1 | rs12076873 | 1 |
| 199747283 | 1 | rs10800786 | 1 |
| 199760987 | 1 | rs6687159 | 1 |
| 199763259 | 1 | rs2152107 | 1 |
| 199763330 | 1 | rs2152106 | 1 |
| 199767008 | 1 | rs17428998 | 1 |
| 199772568 | 1 | rs6689149 | 1 |
| 199842428 | 1 | rs680157 | 1 |
| 178849715 | 2 | rs333995 | 2 |
| 178852274 | 2 | rs333993 | 2 |
| 178853226 | 2 | rs333992 | 2 |
| 178859961 | 2 | rs334630 | 2 |
| 178862909 | 2 | rs7587549 | 2 |
| 178863309 | 2 | rs334623 | 2 |
| 178863697 | 2 | rs334622 | 2 |
| 178865124 | 2 | rs1434080 | 2 |
| 178866571 | 2 | rs334620 | 2 |
| 178868464 | 2 | rs11680778 | 2 |
| 178869031 | 2 | rs13382421 | 2 |
| 178869413 | 2 | rs6756651 | 2 |
| 178872764 | 2 | rs334616 | 2 |
| 178874079 | 2 | rs334614 | 2 |
| 178874406 | 2 | rs13400738 | 2 |
| 178874488 | 2 | rs334613 | 2 |
| 178880063 | 2 | rs334608 | 2 |
| 178881108 | 2 | rs334604 | 2 |
| 178890941 | 2 | rs993598 | 2 |
| 178894775 | 2 | rs7592114 | 2 |
| 178896032 | 2 | rs4133879 | 2 |
| 178898543 | 2 | rs10203141 | 2 |
| 178899497 | 2 | rs11897816 | 2 |
| 178900838 | 2 | rs1434084 | 2 |
| 178908960 | 2 | rs3813253 | 2 |
| 178922038 | 2 | rs1812550 | 2 |
| 178922118 | 2 | rs964165 | 2 |
| 178923399 | 2 | rs1368906 | 2 |
| 178924613 | 2 | rs6433722 | 2 |
| 178934536 | 2 | rs1560871 | 2 |
| 178936378 | 2 | rs1865326 | 2 |
| 178940806 | 2 | rs1434091 | 2 |
| 178941047 | 2 | rs1434092 | 2 |
| 178944214 | 2 | rs12615771 | 2 |
| 178944294 | 2 | rs1434094 | 2 |
| 178945077 | 2 | rs6433724 | 2 |
| 178945596 | 2 | rs10207979 | 2 |
| 178946162 | 2 | rs7607321 | 2 |
| 178946504 | 2 | rs7581278 | 2 |
| 178947087 | 2 | rs10190530 | 2 |
| 178947239 | 2 | rs10167053 | 2 |
| 178947314 | 2 | rs6706354 | 2 |
| 178947934 | 2 | rs6738847 | 2 |
| 178948972 | 2 | rs12476608 | 2 |
| 178949170 | 2 | rs12468466 | 2 |
| 178949298 | 2 | rs12468511 | 2 |
| 178951119 | 2 | rs6724982 | 2 |
| 178951494 | 2 | rs6738749 | 2 |
| 178952860 | 2 | rs1017397 | 2 |
| 178956642 | 2 | rs723841 | 2 |
| 178958643 | 2 | rs2276617 | 2 |
| 178958988 | 2 | rs12473789 | 2 |
| 178959086 | 2 | rs4404314 | 2 |
| 178959252 | 2 | rs728005 | 2 |
| 178966227 | 2 | rs6720559 | 2 |
| 178968628 | 2 | rs2304340 | 2 |
| 178971533 | 2 | rs890579 | 2 |
| 178979129 | 2 | rs4019558 | 2 |
| 178985140 | 2 | rs9288019 | 2 |
| 178989308 | 2 | rs4894017 | 2 |
| 178990683 | 2 | rs7589679 | 2 |
| 178992909 | 2 | rs4894019 | 2 |
| 179002857 | 2 | rs10207436 | 2 |
| 179010130 | 2 | rs2059691 | 2 |
| 179012222 | 2 | rs10930831 | 2 |
| 179031363 | 2 | rs2288322 | 2 |
| 179050199 | 2 | rs6704545 | 2 |
| 179066184 | 2 | rs16866342 | 2 |
| 179069451 | 2 | rs3769866 | 2 |
| 179070127 | 2 | rs12052983 | 2 |
| 179071931 | 2 | rs2249737 | 2 |
| 179074339 | 2 | rs2303537 | 2 |
| 179077162 | 2 | rs3731754 | 2 |
| 179077228 | 2 | rs3820979 | 2 |
| 179091976 | 2 | rs17226357 | 2 |
| 179092663 | 2 | rs957875 | 2 |
| 179104204 | 2 | rs3813250 | 2 |
| 179135432 | 2 | rs2366751 | 2 |
| 179148275 | 2 | rs12464787 | 2 |
| 179151463 | 2 | rs3769864 | 2 |
| 179156094 | 2 | rs4894029 | 2 |
| 179163453 | 2 | rs2163009 | 2 |
| 179169467 | 2 | rs4894030 | 2 |
| 179172772 | 2 | rs1001238 | 2 |
| 179196981 | 2 | rs1017323 | 2 |
| 179248148 | 2 | rs2472751 | 2 |
| 179250144 | 2 | rs2742351 | 2 |
| 179262550 | 2 | rs2244492 | 2 |
| 179290782 | 2 | rs2627043 | 2 |
| 179293638 | 2 | rs2562830 | 2 |
| 179295932 | 2 | rs2742327 | 2 |
| 179357594 | 2 | rs6729746 | 2 |
| 179357995 | 2 | rs3769863 | 2 |
| 179358116 | 2 | rs6733291 | 2 |
| 179358946 | 2 | rs6715406 | 2 |
| 179359199 | 2 | rs6715901 | 2 |
| 179361047 | 2 | rs10497523 | 2 |
| 179375335 | 2 | rs3816849 | 2 |
| 179376051 | 2 | rs3769858 | 2 |
| 179378521 | 2 | rs4894050 | 2 |
| 179380956 | 2 | rs12465459 | 2 |
| 179381609 | 2 | rs11693372 | 2 |
| 179391123 | 2 | rs13006510 | 2 |
| 179393084 | 2 | rs10497526 | 2 |
| 179393228 | 2 | rs1489486 | 2 |
| 179395764 | 2 | rs12471428 | 2 |
| 179396117 | 2 | rs10190741 | 2 |
| 179396700 | 2 | rs1489483 | 2 |
| 179398101 | 2 | rs7561149 | 2 |
| 179398230 | 2 | rs7600330 | 2 |
| 179398478 | 2 | rs6433735 | 2 |
| 179399213 | 2 | rs12464215 | 2 |
| 179399915 | 2 | rs12469367 | 2 |
| 179401212 | 2 | rs7591759 | 2 |
| 179401583 | 2 | rs7592323 | 2 |
| 179401917 | 2 | rs2366911 | 2 |
| 179402020 | 2 | rs2366913 | 2 |
| 179404210 | 2 | rs16866551 | 2 |
| 179404297 | 2 | rs10206931 | 2 |
| 179405660 | 2 | rs11690299 | 2 |
| 179405764 | 2 | rs10199751 | 2 |
| 179410855 | 2 | rs2078403 | 2 |
| 179411647 | 2 | rs1489481 | 2 |
| 179414387 | 2 | rs6732126 | 2 |
| 179414449 | 2 | rs6433737 | 2 |
| 179416792 | 2 | rs1489480 | 2 |
| 179416922 | 2 | rs1489479 | 2 |
| 179419218 | 2 | rs726215 | 2 |
| 179421846 | 2 | rs4894054 | 2 |
| 179421913 | 2 | rs13004438 | 2 |
| 179423841 | 2 | rs2086832 | 2 |
| 179425376 | 2 | rs17362581 | 2 |
| 179425462 | 2 | rs6711319 | 2 |
| 179428211 | 2 | rs1872203 | 2 |
| 179431264 | 2 | rs1027138 | 2 |
| 179432980 | 2 | rs883894 | 2 |
| 179436801 | 2 | rs12052272 | 2 |
| 179437326 | 2 | rs6709708 | 2 |
| 179437586 | 2 | rs12465233 | 2 |
| 179440295 | 2 | rs10185678 | 2 |
| 179440429 | 2 | rs10186056 | 2 |
| 179441225 | 2 | rs10165177 | 2 |
| 179444251 | 2 | rs6731864 | 2 |
| 179445389 | 2 | rs12693170 | 2 |
| 179445855 | 2 | rs12477346 | 2 |
| 179446750 | 2 | rs4894058 | 2 |
| 179447728 | 2 | rs13405116 | 2 |
| 179448254 | 2 | rs6747791 | 2 |
| 179450477 | 2 | rs7565966 | 2 |
| 179454567 | 2 | rs6716304 | 2 |
| 179461794 | 2 | rs1873164 | 2 |
| 179465258 | 2 | rs12693173 | 2 |
| 179466252 | 2 | rs1032282 | 2 |
| 179467679 | 2 | rs6747402 | 2 |
| 179468055 | 2 | rs6750760 | 2 |
| 179470235 | 2 | rs4894062 | 2 |
| 179472729 | 2 | rs1489490 | 2 |
| 179483397 | 2 | rs955738 | 2 |
| 179486662 | 2 | rs6724114 | 2 |
| 179488492 | 2 | rs12468981 | 2 |
| 179488869 | 2 | rs10166147 | 2 |
| 179489100 | 2 | rs12990128 | 2 |
| 179491941 | 2 | rs12467323 | 2 |
| 179495307 | 2 | rs2130220 | 2 |
| 179502692 | 2 | rs10171173 | 2 |
| 179504273 | 2 | rs12990416 | 2 |
| 179504823 | 2 | rs10930843 | 2 |
| 179505816 | 2 | rs10930844 | 2 |
| 179506510 | 2 | rs4894069 | 2 |
| 179509287 | 2 | rs6752606 | 2 |
| 179519149 | 2 | rs2366920 | 2 |
| 179523947 | 2 | rs6433744 | 2 |
| 179524410 | 2 | rs17453452 | 2 |
| 179525451 | 2 | rs7561438 | 2 |
| 179530566 | 2 | rs2200826 | 2 |
| 179533506 | 2 | rs10497528 | 2 |
| 179534084 | 2 | rs17453473 | 2 |
| 179534486 | 2 | rs6725028 | 2 |
| 179537776 | 2 | rs7574599 | 2 |
| 179545295 | 2 | rs6747725 | 2 |
| 179546347 | 2 | rs924800 | 2 |
| 179548078 | 2 | rs10930846 | 2 |
| 179548386 | 2 | rs16866583 | 2 |
| 179550307 | 2 | rs13403584 | 2 |
| 179550864 | 2 | rs10930847 | 2 |
| 179550973 | 2 | rs13008198 | 2 |
| 179551857 | 2 | rs1847420 | 2 |
| 179553719 | 2 | rs4894072 | 2 |
| 179561790 | 2 | rs12993732 | 2 |
| 179568651 | 2 | rs12992925 | 2 |
| 179570641 | 2 | rs11680978 | 2 |
| 179571951 | 2 | rs10930850 | 2 |
| 179575671 | 2 | rs4893862 | 2 |
| 179575971 | 2 | rs11679021 | 2 |
| 179578451 | 2 | rs13025070 | 2 |
| 179579417 | 2 | rs1961416 | 2 |
| 179579620 | 2 | rs4243393 | 2 |
| 179581385 | 2 | rs2886806 | 2 |
| 179597726 | 2 | rs12053571 | 2 |
| 179605032 | 2 | rs2007326 | 2 |
| 179606249 | 2 | rs10930851 | 2 |
| 179608965 | 2 | rs7581299 | 2 |
| 179610345 | 2 | rs2592563 | 2 |
| 179611550 | 2 | rs2655147 | 2 |
| 179612453 | 2 | rs2592560 | 2 |
| 179612878 | 2 | rs2592559 | 2 |
| 179617703 | 2 | rs340340 | 2 |
| 179619662 | 2 | rs1354385 | 2 |
| 179621349 | 2 | rs340337 | 2 |
| 179623639 | 2 | rs340333 | 2 |
| 179624417 | 2 | rs340331 | 2 |
| 8502801 | 3 | rs9879912 | 3 |
| 8502882 | 3 | rs7632954 | 3 |
| 8503431 | 3 | rs7647092 | 3 |
| 8503727 | 3 | rs6791290 | 3 |
| 8505748 | 3 | rs7642129 | 3 |
| 8508268 | 3 | rs1018113 | 3 |
| 8512037 | 3 | rs2047518 | 3 |
| 8521992 | 3 | rs1008003 | 3 |
| 8522741 | 3 | rs9854497 | 3 |
| 8523885 | 3 | rs931686 | 3 |
| 8525015 | 3 | rs7624569 | 3 |
| 8525363 | 3 | rs2167037 | 3 |
| 8528087 | 3 | rs1873944 | 3 |
| 8528238 | 3 | rs9818672 | 3 |
| 8529303 | 3 | rs7622989 | 3 |
| 8530507 | 3 | rs7637683 | 3 |
| 8532559 | 3 | rs3774220 | 3 |
| 8535054 | 3 | rs7616573 | 3 |
| 8538455 | 3 | rs1493586 | 3 |
| 8545426 | 3 | rs9865627 | 3 |
| 8547373 | 3 | rs931517 | 3 |
| 8554850 | 3 | rs6784926 | 3 |
| 8555061 | 3 | rs6776597 | 3 |
| 8561465 | 3 | rs3732987 | 3 |
| 8582533 | 3 | rs1965215 | 3 |
| 8585581 | 3 | rs6764383 | 3 |
| 8586344 | 3 | rs4686268 | 3 |
| 8587703 | 3 | rs355133 | 3 |
| 8588499 | 3 | rs17728877 | 3 |
| 8593305 | 3 | rs165174 | 3 |
| 8593504 | 3 | rs4686269 | 3 |
| 8595174 | 3 | rs355137 | 3 |
| 8596493 | 3 | rs956376 | 3 |
| 8596720 | 3 | rs6765903 | 3 |
| 8598342 | 3 | rs1876609 | 3 |
| 8600917 | 3 | rs11131138 | 3 |
| 8606704 | 3 | rs4686277 | 3 |
| 8608172 | 3 | rs355124 | 3 |
| 8612274 | 3 | rs355126 | 3 |
| 8620707 | 3 | rs9865570 | 3 |
| 8621870 | 3 | rs355091 | 3 |
| 8624260 | 3 | rs903115 | 3 |
| 8624462 | 3 | rs2324663 | 3 |
| 8635008 | 3 | rs903116 | 3 |
| 8644915 | 3 | rs1123067 | 3 |
| 8659764 | 3 | rs164956 | 3 |
| 8660801 | 3 | rs164958 | 3 |
| 8661191 | 3 | rs164959 | 3 |
| 8662378 | 3 | rs4686283 | 3 |
| 8662453 | 3 | rs4684602 | 3 |
| 8669057 | 3 | rs3774209 | 3 |
| 8669907 | 3 | rs403412 | 3 |
| 8671316 | 3 | rs420867 | 3 |
| 8672372 | 3 | rs6785267 | 3 |
| 8686575 | 3 | rs11916383 | 3 |
| 8687227 | 3 | rs164954 | 3 |
| 8710542 | 3 | rs164458 | 3 |
| 8720008 | 3 | rs4053823 | 3 |
| 8723142 | 3 | rs164461 | 3 |
| 8723854 | 3 | rs164463 | 3 |
| 8724171 | 3 | rs164466 | 3 |
| 8724558 | 3 | rs164467 | 3 |
| 8727987 | 3 | rs6443200 | 3 |
| 8728218 | 3 | rs6443202 | 3 |
| 8731772 | 3 | rs11718527 | 3 |
| 8739300 | 3 | rs1873035 | 3 |
| 8748131 | 3 | rs237860 | 3 |
| 8748285 | 3 | rs237862 | 3 |
| 8748968 | 3 | rs237865 | 3 |
| 8749129 | 3 | rs237866 | 3 |
| 8755438 | 3 | rs237872 | 3 |
| 8757437 | 3 | rs237876 | 3 |
| 8763198 | 3 | rs11476 | 3 |
| 8769545 | 3 | rs1042778 | 3 |
| 8772042 | 3 | rs237887 | 3 |
| 8775672 | 3 | rs2268492 | 3 |
| 8777483 | 3 | rs237889 | 3 |
| 8789012 | 3 | rs401015 | 3 |
| 8791344 | 3 | rs237922 | 3 |
| 8808878 | 3 | rs237852 | 3 |
| 8809723 | 3 | rs237851 | 3 |
| 8823916 | 3 | rs1714299 | 3 |
| 8829427 | 3 | rs759772 | 3 |
| 8849963 | 3 | rs345102 | 3 |
| 8856649 | 3 | rs124844 | 3 |
| 8858040 | 3 | rs2270474 | 3 |
| 8859136 | 3 | rs7646345 | 3 |
| 8869415 | 3 | rs6785302 | 3 |
| 8869856 | 3 | rs1005989 | 3 |
| 8872165 | 3 | rs4686310 | 3 |
| 8872236 | 3 | rs4684614 | 3 |
| 8985394 | 3 | rs555780 | 3 |
| 8991057 | 3 | rs689290 | 3 |
| 9001818 | 3 | rs186889 | 3 |
| 9004485 | 3 | rs341784 | 3 |
| 9007897 | 3 | rs583509 | 3 |
| 9009558 | 3 | rs511815 | 3 |
| 12350391 | 3 | rs1122648 | 4 |
| 12351745 | 3 | rs17036314 | 4 |
| 12372392 | 3 | rs17817276 | 4 |
| 12377474 | 3 | rs1373641 | 4 |
| 12387115 | 3 | rs2921188 | 4 |
| 12393612 | 3 | rs2028759 | 4 |
| 12404468 | 3 | rs2938395 | 4 |
| 12409608 | 3 | rs2938392 | 4 |
| 12417731 | 3 | rs2959273 | 4 |
| 12417833 | 3 | rs2959272 | 4 |
| 12420807 | 3 | rs1151996 | 4 |
| 12421004 | 3 | rs1151997 | 4 |
| 12421492 | 3 | rs1151998 | 4 |
| 12422153 | 3 | rs1151999 | 4 |
| 12424528 | 3 | rs796313 | 4 |
| 12424683 | 3 | rs796290 | 4 |
| 12425354 | 3 | rs709149 | 4 |
| 12426337 | 3 | rs709150 | 4 |
| 12429999 | 3 | rs709151 | 4 |
| 12431834 | 3 | rs709154 | 4 |
| 12437024 | 3 | rs709157 | 4 |
| 12438826 | 3 | rs7645903 | 4 |
| 12440243 | 3 | rs1175540 | 4 |
| 12440488 | 3 | rs1175541 | 4 |
| 12441214 | 3 | rs1175542 | 4 |
| 12442044 | 3 | rs1175544 | 4 |
| 12443463 | 3 | rs13099634 | 4 |
| 12445239 | 3 | rs1797912 | 4 |
| 12452055 | 3 | rs1152003 | 4 |
| 12456375 | 3 | rs4498025 | 4 |
| 12458104 | 3 | rs1152004 | 4 |
| 12461421 | 3 | rs1184332 | 4 |
| 12462047 | 3 | rs1152006 | 4 |
| 12462547 | 3 | rs1152007 | 4 |
| 12462612 | 3 | rs1152008 | 4 |
| 12462991 | 3 | rs7618026 | 4 |
| 12467949 | 3 | rs13064115 | 4 |
| 12468347 | 3 | rs9855622 | 4 |
| 12471461 | 3 | rs9872031 | 4 |
| 12477955 | 3 | rs709167 | 4 |
| 12479295 | 3 | rs1699346 | 4 |
| 12480160 | 3 | rs3936555 | 4 |
| 12481675 | 3 | rs11917039 | 4 |
| 12482401 | 3 | rs17036788 | 4 |
| 12482564 | 3 | rs1699348 | 4 |
| 12489157 | 3 | rs709164 | 4 |
| 12496698 | 3 | rs1797886 | 4 |
| 12501402 | 3 | rs709160 | 4 |
| 12505828 | 3 | rs905644 | 4 |
| 12507057 | 3 | rs299640 | 4 |
| 12508335 | 3 | rs299639 | 4 |
| 12509327 | 3 | rs299638 | 4 |
| 12513719 | 3 | rs299649 | 4 |
| 12513926 | 3 | rs299648 | 4 |
| 12521172 | 3 | rs2596827 | 4 |
| 12522657 | 3 | rs299652 | 4 |
| 12523943 | 3 | rs299653 | 4 |
| 12526491 | 3 | rs299657 | 4 |
| 12529069 | 3 | rs299661 | 4 |
| 12534386 | 3 | rs1797889 | 4 |
| 12535763 | 3 | rs1618545 | 4 |
| 12538640 | 3 | rs2655260 | 4 |
| 12539033 | 3 | rs796174 | 4 |
| 12539302 | 3 | rs796173 | 4 |
| 12539536 | 3 | rs796172 | 4 |
| 12543425 | 3 | rs299630 | 4 |
| 12548518 | 3 | rs299642 | 4 |
| 12551846 | 3 | rs299629 | 4 |
| 12554944 | 3 | rs1466835 | 4 |
| 12555283 | 3 | rs1552485 | 4 |
| 12561240 | 3 | rs2454435 | 4 |
| 12573963 | 3 | rs2255648 | 4 |
| 12577494 | 3 | rs2450855 | 4 |
| 12582929 | 3 | rs2454427 | 4 |
| 12584937 | 3 | rs2633442 | 4 |
| 12585074 | 3 | rs2442803 | 4 |
| 12586269 | 3 | rs1962515 | 4 |
| 12590580 | 3 | rs2442804 | 4 |
| 12592333 | 3 | rs2648309 | 4 |
| 12592996 | 3 | rs1542848 | 4 |
| 12597623 | 3 | rs9849171 | 4 |
| 12597943 | 3 | rs2596825 | 4 |
| 12599070 | 3 | rs15997 | 4 |
| 12601516 | 3 | rs3729931 | 4 |
| 12603920 | 3 | rs2290159 | 4 |
| 12607652 | 3 | rs2596831 | 4 |
| 12605083 | 3 | rs2290160 | 4 |
| 12619335 | 3 | rs1039244 | 4 |
| 12621255 | 3 | rs6777175 | 4 |
| 12622547 | 3 | rs17771249 | 4 |
| 12624857 | 3 | rs2055311 | 4 |
| 12624937 | 3 | rs963959 | 4 |
| 12628233 | 3 | rs3730269 | 4 |
| 12628800 | 3 | rs5746195 | 4 |
| 12630689 | 3 | rs9809501 | 4 |
| 12634494 | 3 | rs4684867 | 4 |
| 12637370 | 3 | rs11705805 | 4 |
| 12638835 | 3 | rs11923427 | 4 |
| 12642784 | 3 | rs11711419 | 4 |
| 12649566 | 3 | rs12634077 | 4 |
| 12651113 | 3 | rs9817675 | 4 |
| 12653725 | 3 | rs7636754 | 4 |
| 12664858 | 3 | rs6442322 | 4 |
| 12672498 | 3 | rs4684871 | 4 |
| 12677562 | 3 | rs6795441 | 4 |
| 12679894 | 3 | rs904453 | 4 |
| 12684167 | 3 | rs7617041 | 4 |
| 12692321 | 3 | rs13061104 | 4 |
| 12695967 | 3 | rs9874557 | 4 |
| 12702962 | 3 | rs9874315 | 4 |
| 12704485 | 3 | rs4684111 | 4 |
| 12704887 | 3 | rs6792154 | 4 |
| 12711530 | 3 | rs9818046 | 4 |
| 12722915 | 3 | rs9811396 | 4 |
| 12724130 | 3 | rs2030151 | 4 |
| 12731946 | 3 | rs12637499 | 4 |
| 12742194 | 3 | rs7644528 | 4 |
| 12754409 | 3 | rs2271209 | 4 |
| 12778792 | 3 | rs9876483 | 4 |
| 12780934 | 3 | rs9831765 | 4 |
| 12781080 | 3 | rs7626609 | 4 |
| 12781142 | 3 | rs7638333 | 4 |
| 12793205 | 3 | rs9813960 | 4 |
| 12808079 | 3 | rs9874893 | 4 |
| 12814617 | 3 | rs4535210 | 4 |
| 12815934 | 3 | rs6810325 | 4 |
| 12816804 | 3 | rs7650482 | 4 |
| 12817932 | 3 | rs9820977 | 4 |
| 12820158 | 3 | rs9835677 | 4 |
| 12823822 | 3 | rs11718898 | 4 |
| 12832493 | 3 | rs2305397 | 4 |
| 12836586 | 3 | rs4684883 | 4 |
| 12839518 | 3 | rs6800812 | 4 |
| 12844690 | 3 | rs6442335 | 4 |
| 12846267 | 3 | rs7612690 | 4 |
| 12846866 | 3 | rs1876617 | 4 |
| 12846997 | 3 | rs11914341 | 4 |
| 12849340 | 3 | rs6442338 | 4 |
| 12850443 | 3 | rs12629133 | 4 |
| 12850880 | 3 | rs1079361 | 4 |
| 12853736 | 3 | rs3773306 | 4 |
| 12855030 | 3 | rs3821608 | 4 |
| 12855818 | 3 | rs2290312 | 4 |
| 12856757 | 3 | rs7627602 | 4 |
| 12867850 | 3 | rs7645477 | 4 |
| 12874890 | 3 | rs7645264 | 4 |
| 12875288 | 3 | rs7637490 | 4 |
| 12875901 | 3 | rs1810077 | 4 |
| 12886230 | 3 | rs361220 | 4 |
| 12887765 | 3 | rs17612927 | 4 |
| 12888536 | 3 | rs187250 | 4 |
| 12891433 | 3 | rs693201 | 4 |
| 12891608 | 3 | rs393862 | 4 |
| 12892207 | 3 | rs434460 | 4 |
| 12894988 | 3 | rs361226 | 4 |
| 12895171 | 3 | rs361225 | 4 |
| 12900190 | 3 | rs2082511 | 4 |
| 12903548 | 3 | rs2569994 | 4 |
| 12903762 | 3 | rs2600226 | 4 |
| 12908866 | 3 | rs2600224 | 4 |
| 12914546 | 3 | rs9211 | 4 |
| 12918608 | 3 | rs4547681 | 4 |
| 12920658 | 3 | rs892281 | 4 |
| 46624704 | 3 | rs7640056 | 5 |
| 46627433 | 3 | rs6442009 | 5 |
| 46634864 | 3 | rs869701 | 5 |
| 46635440 | 3 | rs2385858 | 5 |
| 46636930 | 3 | rs11130098 | 5 |
| 46637740 | 3 | rs6773430 | 5 |
| 46643693 | 3 | rs1356875 | 5 |
| 46650490 | 3 | rs7643859 | 5 |
| 46651593 | 3 | rs6805749 | 5 |
| 46652463 | 3 | rs1520488 | 5 |
| 46654679 | 3 | rs4234464 | 5 |
| 46655067 | 3 | rs11709020 | 5 |
| 46655487 | 3 | rs11709936 | 5 |
| 46656377 | 3 | rs13083665 | 5 |
| 46658381 | 3 | rs950190 | 5 |
| 46660726 | 3 | rs6442011 | 5 |
| 46663566 | 3 | rs7633770 | 5 |
| 46664571 | 3 | rs11130102 | 5 |
| 46673542 | 3 | rs4683259 | 5 |
| 46676118 | 3 | rs7373994 | 5 |
| 46677475 | 3 | rs877680 | 5 |
| 46680373 | 3 | rs4682826 | 5 |
| 46686511 | 3 | rs3827495 | 5 |
| 46686811 | 3 | rs11711100 | 5 |
| 46688869 | 3 | rs3935248 | 5 |
| 46690005 | 3 | rs4682825 | 5 |
| 46691527 | 3 | rs7433347 | 5 |
| 46692496 | 3 | rs4683269 | 5 |
| 46692935 | 3 | rs6808525 | 5 |
| 46696322 | 3 | rs6442019 | 5 |
| 46696523 | 3 | rs10451990 | 5 |
| 46696666 | 3 | rs6803988 | 5 |
| 46703212 | 3 | rs7373358 | 5 |
| 46703908 | 3 | rs7426833 | 5 |
| 46705067 | 3 | rs12496585 | 5 |
| 46705753 | 3 | rs4076927 | 5 |
| 46709445 | 3 | rs11718902 | 5 |
| 46710649 | 3 | rs6442020 | 5 |
| 46717023 | 3 | rs6442021 | 5 |
| 46723457 | 3 | rs12491225 | 5 |
| 46728980 | 3 | rs9815362 | 5 |
| 46753012 | 3 | rs6793661 | 5 |
| 46753229 | 3 | rs12492369 | 5 |
| 46753846 | 3 | rs12493158 | 5 |
| 46755307 | 3 | rs6797748 | 5 |
| 46759906 | 3 | rs6799447 | 5 |
| 46771793 | 3 | rs6773559 | 5 |
| 46777439 | 3 | rs6442028 | 5 |
| 46848961 | 3 | rs7636089 | 5 |
| 46888341 | 3 | rs7621046 | 5 |
| 46900543 | 3 | rs11926707 | 5 |
| 46904550 | 3 | rs6442037 | 5 |
| 46910685 | 3 | rs724449 | 5 |
| 46926939 | 3 | rs11719795 | 5 |
| 46930408 | 3 | rs4682845 | 5 |
| 46935801 | 3 | rs6442039 | 5 |
| 46940201 | 3 | rs2290546 | 5 |
| 46940860 | 3 | rs6780601 | 5 |
| 46941391 | 3 | rs4683298 | 5 |
| 46942044 | 3 | rs2290545 | 5 |
| 46948677 | 3 | rs4683297 | 5 |
| 46948824 | 3 | rs4683296 | 5 |
| 46954017 | 3 | rs4683294 | 5 |
| 46963565 | 3 | rs13092573 | 5 |
| 46969991 | 3 | rs1463393 | 5 |
| 46976047 | 3 | rs12630534 | 5 |
| 46984809 | 3 | rs11130111 | 5 |
| 46986124 | 3 | rs13077398 | 5 |
| 46988223 | 3 | rs17784031 | 5 |
| 46999499 | 3 | rs11130112 | 5 |
| 47001889 | 3 | rs4234465 | 5 |
| 47002142 | 3 | rs4682831 | 5 |
| 47006437 | 3 | rs10433549 | 5 |
| 47010904 | 3 | rs749512 | 5 |
| 47018542 | 3 | rs2305634 | 5 |
| 47020743 | 3 | rs1079276 | 5 |
| 47020850 | 3 | rs2305637 | 5 |
| 47029873 | 3 | rs2278963 | 5 |
| 47036704 | 3 | rs17784127 | 5 |
| 47039440 | 3 | rs7610636 | 5 |
| 47045501 | 3 | rs11917361 | 5 |
| 47049910 | 3 | rs11713693 | 5 |
| 47061052 | 3 | rs9858443 | 5 |
| 47083334 | 3 | rs9844122 | 5 |
| 47089908 | 3 | rs6794193 | 5 |
| 47095644 | 3 | rs7433300 | 5 |
| 47100389 | 3 | rs4082155 | 5 |
| 47113712 | 3 | rs13061071 | 5 |
| 47113882 | 3 | rs9311403 | 5 |
| 150640450 | 7 | rs378140 | 6 |
| 150662440 | 7 | rs7807594 | 6 |
| 150674633 | 7 | rs438559 | 6 |
| 150678945 | 7 | rs404919 | 6 |
| 150679417 | 7 | rs449081 | 6 |
| 150680164 | 7 | rs366355 | 6 |
| 150680436 | 7 | rs386223 | 6 |
| 150683230 | 7 | rs451593 | 6 |
| 150684566 | 7 | rs11771465 | 6 |
| 150700344 | 7 | rs2108726 | 6 |
| 150705229 | 7 | rs2159158 | 6 |
| 150707507 | 7 | rs7795959 | 6 |
| 150715009 | 7 | rs732980 | 6 |
| 150717555 | 7 | rs11769160 | 6 |
| 150717730 | 7 | rs1558164 | 6 |
| 150719218 | 7 | rs4726022 | 6 |
| 150722823 | 7 | rs11760463 | 6 |
| 150724981 | 7 | rs11761588 | 6 |
| 150730567 | 7 | rs1004340 | 6 |
| 150732477 | 7 | rs887406 | 6 |
| 150739344 | 7 | rs917613 | 6 |
| 150746624 | 7 | rs6945096 | 6 |
| 150753436 | 7 | rs7806340 | 6 |
| 150754462 | 7 | rs7792547 | 6 |
| 150754699 | 7 | rs7792955 | 6 |
| 150755021 | 7 | rs7793301 | 6 |
| 150766436 | 7 | rs12539860 | 6 |
| 150771439 | 7 | rs6464143 | 6 |
| 150773869 | 7 | rs12537413 | 6 |
| 150778055 | 7 | rs7784512 | 6 |
| 150783598 | 7 | rs9640291 | 6 |
| 150787262 | 7 | rs4368893 | 6 |
| 150800952 | 7 | rs1109089 | 6 |
| 150803014 | 7 | rs7805967 | 6 |
| 150803215 | 7 | rs17713697 | 6 |
| 150808067 | 7 | rs2299962 | 6 |
| 150823595 | 7 | rs2299965 | 6 |
| 150826261 | 7 | rs11772458 | 6 |
| 150837159 | 7 | rs3827805 | 6 |
| 150840324 | 7 | rs4298422 | 6 |
| 150843157 | 7 | rs12112134 | 6 |
| 150851574 | 7 | rs7794922 | 6 |
| 150865170 | 7 | rs11771194 | 6 |
| 150873904 | 7 | rs12703139 | 6 |
| 150875430 | 7 | rs1029955 | 6 |
| 150881337 | 7 | rs12703143 | 6 |
| 150882266 | 7 | rs4726043 | 6 |
| 150883341 | 7 | rs6952042 | 6 |
| 150884016 | 7 | rs4726046 | 6 |
| 150885108 | 7 | rs8961 | 6 |
| 150886485 | 7 | rs13237561 | 6 |
| 150887065 | 7 | rs4726050 | 6 |
| 150887906 | 7 | rs7806619 | 6 |
| 150906805 | 7 | rs1029945 | 6 |
| 150929179 | 7 | rs2727572 | 6 |
| 150939500 | 7 | rs4726060 | 6 |
| 150942427 | 7 | rs2374229 | 6 |
| 150950315 | 7 | rs2536059 | 6 |
| 150950488 | 7 | rs6464156 | 6 |
| 150959151 | 7 | rs4726070 | 6 |
| 150978395 | 7 | rs2727527 | 6 |
| 150982165 | 7 | rs6964824 | 6 |
| 150982449 | 7 | rs2058394 | 6 |
| 150987811 | 7 | rs2254644 | 6 |
| 150988633 | 7 | rs6464160 | 6 |
| 150995290 | 7 | rs1105843 | 6 |
| 151000719 | 7 | rs2727535 | 6 |
| 151004475 | 7 | rs867668 | 6 |
| 151011519 | 7 | rs6464161 | 6 |
| 151016184 | 7 | rs2727549 | 6 |
| 151017330 | 7 | rs2536086 | 6 |
| 151018897 | 7 | rs2024266 | 6 |
| 151026282 | 7 | rs7780804 | 6 |
| 151027904 | 7 | rs11983465 | 6 |
| 151029032 | 7 | rs6968460 | 6 |
| 151033723 | 7 | rs2374270 | 6 |
| 151034062 | 7 | rs2727563 | 6 |
| 151040039 | 7 | rs2536077 | 6 |
| 151045974 | 7 | rs10224002 | 6 |
| 151056382 | 7 | rs2536067 | 6 |
| 151067285 | 7 | rs885273 | 6 |
| 151074864 | 7 | rs1860741 | 6 |
| 151075708 | 7 | rs7788160 | 6 |
| 151081309 | 7 | rs7807769 | 6 |
| 151083561 | 7 | rs7801616 | 6 |
| 151135432 | 7 | rs10257529 | 6 |
| 151136023 | 7 | rs7797865 | 6 |
| 151137088 | 7 | rs6464173 | 6 |
| 151139777 | 7 | rs1881638 | 6 |
| 151140899 | 7 | rs11773011 | 6 |
| 151143782 | 7 | rs11768925 | 6 |
| 151147009 | 7 | rs6975614 | 6 |
| 151147753 | 7 | rs1881629 | 6 |
| 151148772 | 7 | rs13309585 | 6 |
| 151148940 | 7 | rs13310994 | 6 |
| 151151192 | 7 | rs12703162 | 6 |
| 151154106 | 7 | rs17567084 | 6 |
| 151157024 | 7 | rs11760502 | 6 |
| 151168718 | 7 | rs9648730 | 6 |
| 151171566 | 7 | rs10277544 | 6 |
| 151171630 | 7 | rs10277655 | 6 |
| 151172250 | 7 | rs10236110 | 6 |
| 151175924 | 7 | rs11772236 | 6 |
| 151188776 | 7 | rs7789674 | 6 |
| 151189930 | 7 | rs9640300 | 6 |
| 151197847 | 7 | rs13240743 | 6 |
| 151211581 | 7 | rs12540735 | 6 |
| 151218976 | 7 | rs10215172 | 6 |
| 151223649 | 7 | rs10952325 | 6 |
| 151225840 | 7 | rs7788961 | 6 |
| 151227390 | 7 | rs2374272 | 6 |
| 151229489 | 7 | rs2374273 | 6 |
| 151229858 | 7 | rs4272280 | 6 |
| 151237399 | 7 | rs17490405 | 6 |
| 151264198 | 7 | rs10807990 | 6 |
| 151267242 | 7 | rs10952333 | 6 |
| 151269685 | 7 | rs12535522 | 6 |
| 151274976 | 7 | rs2374315 | 6 |
| 151276945 | 7 | rs10233081 | 6 |
| 151278559 | 7 | rs10267553 | 6 |
| 151278747 | 7 | rs7807816 | 6 |
| 151279887 | 7 | rs7794520 | 6 |
| 151281173 | 7 | rs11766254 | 6 |
| 151283644 | 7 | rs7797169 | 6 |
| 151284568 | 7 | rs10952334 | 6 |
| 151285905 | 7 | rs13240848 | 6 |
| 151286572 | 7 | rs10952335 | 6 |
| 151289681 | 7 | rs6979879 | 6 |
| 151293117 | 7 | rs10278073 | 6 |
| 151298378 | 7 | rs4726119 | 6 |
| 151299368 | 7 | rs7799325 | 6 |
| 151305320 | 7 | rs1544031 | 6 |
| 151306403 | 7 | rs4726123 | 6 |
| 151308578 | 7 | rs10464436 | 6 |
| 151312755 | 7 | rs1406522 | 6 |
| 151317580 | 7 | rs12670289 | 6 |
| 151324435 | 7 | rs11766982 | 6 |
| 151325039 | 7 | rs6948803 | 6 |
| 151325419 | 7 | rs4236435 | 6 |
| 151327965 | 7 | rs11765654 | 6 |
| 151328127 | 7 | rs7797930 | 6 |
| 151329306 | 7 | rs10251856 | 6 |
| 151334220 | 7 | rs4726130 | 6 |
| 151337042 | 7 | rs4726132 | 6 |
| 151341480 | 7 | rs1019173 | 6 |
| 151347382 | 7 | rs7808913 | 6 |
| 46312508 | 11 | rs11038866 | 7 |
| 46478882 | 11 | rs7949282 | 7 |
| 46491122 | 11 | rs3802890 | 7 |
| 46500100 | 11 | rs11038910 | 7 |
| 46614006 | 11 | rs6485686 | 7 |
| 46717332 | 11 | rs3136516 | 7 |
| 46855347 | 11 | rs6485702 | 7 |
| 46955523 | 11 | rs4752817 | 7 |
| 46958484 | 11 | rs7130812 | 7 |
| 47027001 | 11 | rs17787966 | 7 |
| 47038308 | 11 | rs11039074 | 7 |
| 47040637 | 11 | rs10769234 | 7 |
| 47057372 | 11 | rs7946709 | 7 |
| 47073399 | 11 | rs7112854 | 7 |
| 47083729 | 11 | rs7117404 | 7 |
| 47085973 | 11 | rs12417519 | 7 |
| 47086430 | 11 | rs4587689 | 7 |
| 47087354 | 11 | rs10838658 | 7 |
| 47089300 | 11 | rs12796744 | 7 |
| 47108849 | 11 | rs10838660 | 7 |
| 47114441 | 11 | rs11039106 | 7 |
| 47120784 | 11 | rs923324 | 7 |
| 47130638 | 11 | rs10501319 | 7 |
| 47130937 | 11 | rs12224096 | 7 |
| 47136405 | 11 | rs1060573 | 7 |
| 47142086 | 11 | rs4752969 | 7 |
| 47142256 | 11 | rs11039114 | 7 |
| 47143000 | 11 | rs1055447 | 7 |
| 47145168 | 11 | rs3740690 | 7 |
| 47150142 | 11 | rs11600668 | 7 |
| 47153558 | 11 | rs2279439 | 7 |
| 47159522 | 11 | rs7925137 | 7 |
| 47160751 | 11 | rs2279438 | 7 |
| 47166048 | 11 | rs901750 | 7 |
| 47185892 | 11 | rs11039130 | 7 |
| 47191294 | 11 | rs2029298 | 7 |
| 47200241 | 11 | rs1685404 | 7 |
| 47205870 | 11 | rs2957873 | 7 |
| 47212174 | 11 | rs326224 | 7 |
| 47217836 | 11 | rs11988 | 7 |
| 47225126 | 11 | rs7129661 | 7 |
| 47225735 | 11 | rs4752973 | 7 |
| 47227351 | 11 | rs3758668 | 7 |
| 47235514 | 11 | rs3758674 | 7 |
| 47254936 | 11 | rs326214 | 7 |
| 47259309 | 11 | rs11606683 | 7 |
| 47259851 | 11 | rs326217 | 7 |
| 47263161 | 11 | rs1051006 | 7 |
| 47269468 | 11 | rs10838687 | 7 |
| 47276282 | 11 | rs17726390 | 7 |
| 47302492 | 11 | rs2290149 | 7 |
| 47303299 | 11 | rs11039182 | 7 |
| 47306545 | 11 | rs753993 | 7 |
| 47307129 | 11 | rs10838693 | 7 |
| 47318915 | 11 | rs10769253 | 7 |
| 47323947 | 11 | rs10769255 | 7 |
| 47328953 | 11 | rs2071304 | 7 |
| 47333024 | 11 | rs1057233 | 7 |
| 47337169 | 11 | rs3740689 | 7 |
| 47342499 | 11 | rs10838698 | 7 |
| 47353230 | 11 | rs4752829 | 7 |
| 47357747 | 11 | rs11601603 | 7 |
| 47360890 | 11 | rs11828339 | 7 |
| 47366742 | 11 | rs11821917 | 7 |
| 47366969 | 11 | rs4752990 | 7 |
| 47367527 | 11 | rs4752993 | 7 |
| 47368157 | 11 | rs10769262 | 7 |
| 47385141 | 11 | rs4752999 | 7 |
| 47388105 | 11 | rs753812 | 7 |
| 47388879 | 11 | rs755553 | 7 |
| 47396514 | 11 | rs10742803 | 7 |
| 47398089 | 11 | rs10838708 | 7 |
| 47403778 | 11 | rs17790804 | 7 |
| 47410847 | 11 | rs4237544 | 7 |
| 47414115 | 11 | rs11604680 | 7 |
| 47418359 | 11 | rs7103648 | 7 |
| 47434425 | 11 | rs17198158 | 7 |
| 47443461 | 11 | rs1317149 | 7 |
| 47465713 | 11 | rs7933019 | 7 |
| 47486523 | 11 | rs7124681 | 7 |
| 47486600 | 11 | rs6485758 | 7 |
| 47488460 | 11 | rs4752843 | 7 |
| 47488971 | 11 | rs11039266 | 7 |
| 47496273 | 11 | rs4752845 | 7 |
| 47510913 | 11 | rs10742814 | 7 |
| 47521679 | 11 | rs12285933 | 7 |
| 47581290 | 11 | rs12419692 | 7 |
| 47583878 | 11 | rs6485763 | 7 |
| 47600467 | 11 | rs3817335 | 7 |
| 47607569 | 11 | rs3817334 | 7 |
| 47615711 | 11 | rs7118178 | 7 |
| 47619508 | 11 | rs7120548 | 7 |
| 47625865 | 11 | rs7107922 | 7 |
| 47636556 | 11 | rs10838740 | 7 |
| 47645919 | 11 | rs10742817 | 7 |
| 47660519 | 11 | rs3758655 | 7 |
| 47662867 | 11 | rs10838746 | 7 |
| 47709351 | 11 | rs17788930 | 7 |
| 47719592 | 11 | rs10838757 | 7 |
| 47756351 | 11 | rs9909 | 7 |
| 47757677 | 11 | rs7114813 | 7 |
| 47782666 | 11 | rs11039402 | 7 |
| 47787908 | 11 | rs10838771 | 7 |
| 47792878 | 11 | rs7131262 | 7 |
| 47793984 | 11 | rs6485786 | 7 |
| 47798438 | 11 | rs4752796 | 7 |
| 47822136 | 11 | rs10769300 | 7 |
| 47842940 | 11 | rs4752798 | 7 |
| 47859459 | 11 | rs12364432 | 7 |
| 47864217 | 11 | rs4752801 | 7 |
| 47868726 | 11 | rs1471712 | 7 |
| 47886422 | 11 | rs6485795 | 7 |
| 48048265 | 11 | rs7395231 | 7 |
| 48051345 | 11 | rs10769312 | 7 |
| 48051455 | 11 | rs10838800 | 7 |
| 48054752 | 11 | rs1039485 | 7 |
| 48059054 | 11 | rs10838802 | 7 |
| 48059443 | 11 | rs6485807 | 7 |
| 48063062 | 11 | rs7122335 | 7 |
| 48074054 | 11 | rs1503188 | 7 |
| 48075569 | 11 | rs10838805 | 7 |
| 48100800 | 11 | rs2870080 | 7 |
| 48102322 | 11 | rs11039530 | 7 |
| 48114445 | 11 | rs2270994 | 7 |
| 48118881 | 11 | rs7124275 | 7 |
| 48122843 | 11 | rs4752904 | 7 |
| 48124314 | 11 | rs7947811 | 7 |
| 48124679 | 11 | rs11039543 | 7 |
| 48132407 | 11 | rs10742836 | 7 |
| 48138813 | 11 | rs1039481 | 7 |
| 48143754 | 11 | rs10838814 | 7 |
| 48156473 | 11 | rs2047815 | 7 |
| 48189429 | 11 | rs1393794 | 7 |
| 48196688 | 11 | rs11039593 | 7 |
| 48200883 | 11 | rs1503186 | 7 |
| 48235992 | 11 | rs1393788 | 7 |
| 48240609 | 11 | rs1109906 | 7 |
| 48241877 | 11 | rs713325 | 7 |
| 48250507 | 11 | rs11039627 | 7 |
| 48251879 | 11 | rs7479393 | 7 |
| 48283349 | 11 | rs1483123 | 7 |
| 48288604 | 11 | rs12282928 | 7 |
| 48297384 | 11 | rs12364360 | 7 |
| 48300235 | 11 | rs1872023 | 7 |
| 48301955 | 11 | rs7946068 | 7 |
| 48303716 | 11 | rs10838874 | 7 |
| 48310420 | 11 | rs7395538 | 7 |
| 109591853 | 12 | rs16940935 | 8 |
| 109599496 | 12 | rs871921 | 8 |
| 109601038 | 12 | rs2339524 | 8 |
| 109605685 | 12 | rs1018133 | 8 |
| 109620842 | 12 | rs16940950 | 8 |
| 109622543 | 12 | rs11065698 | 8 |
| 109639914 | 12 | rs11065706 | 8 |
| 109640330 | 12 | rs10774603 | 8 |
| 109642544 | 12 | rs1050587 | 8 |
| 109644386 | 12 | rs1973505 | 8 |
| 109648620 | 12 | rs7960552 | 8 |
| 109648798 | 12 | rs7960761 | 8 |
| 109649498 | 12 | rs16940970 | 8 |
| 109655409 | 12 | rs7301769 | 8 |
| 109660596 | 12 | rs1107652 | 8 |
| 109679793 | 12 | rs12300445 | 8 |
| 109680587 | 12 | rs9300316 | 8 |
| 109685020 | 12 | rs9668929 | 8 |
| 109691898 | 12 | rs16940992 | 8 |
| 109697095 | 12 | rs17683336 | 8 |
| 109699958 | 12 | rs17793540 | 8 |
| 109703823 | 12 | rs650518 | 8 |
| 109705791 | 12 | rs1123852 | 8 |
| 109714028 | 12 | rs850517 | 8 |
| 109724204 | 12 | rs850511 | 8 |
| 109726744 | 12 | rs850509 | 8 |
| 109731023 | 12 | rs7974845 | 8 |
| 109733007 | 12 | rs11065724 | 8 |
| 109733160 | 12 | rs6489954 | 8 |
| 109735485 | 12 | rs7963064 | 8 |
| 109738267 | 12 | rs960669 | 8 |
| 109744939 | 12 | rs1858886 | 8 |
| 109751414 | 12 | rs1476470 | 8 |
| 109756870 | 12 | rs2188398 | 8 |
| 109756936 | 12 | rs2188399 | 8 |
| 109759110 | 12 | rs10849910 | 8 |
| 109763724 | 12 | rs876373 | 8 |
| 109764987 | 12 | rs2238147 | 8 |
| 109769404 | 12 | rs2339636 | 8 |
| 109770477 | 12 | rs7980320 | 8 |
| 109772109 | 12 | rs2283348 | 8 |
| 109774077 | 12 | rs1858887 | 8 |
| 109775254 | 12 | rs886132 | 8 |
| 109778903 | 12 | rs11065733 | 8 |
| 109792988 | 12 | rs4766513 | 8 |
| 109796312 | 12 | rs2238149 | 8 |
| 109798672 | 12 | rs11612727 | 8 |
| 109801077 | 12 | rs1468131 | 8 |
| 109807791 | 12 | rs10849913 | 8 |
| 109809648 | 12 | rs10774609 | 8 |
| 109818276 | 12 | rs17191922 | 8 |
| 109818701 | 12 | rs6489821 | 8 |
| 109824626 | 12 | rs10774610 | 8 |
| 109828713 | 12 | rs1011300 | 8 |
| 109834232 | 12 | rs11065766 | 8 |
| 109835382 | 12 | rs2233260 | 8 |
| 109837231 | 12 | rs933296 | 8 |
| 109837544 | 12 | rs17192160 | 8 |
| 109844095 | 12 | rs4766517 | 8 |
| 109845681 | 12 | rs12828640 | 8 |
| 109847911 | 12 | rs7957842 | 8 |
| 109849233 | 12 | rs756823 | 8 |
| 109849707 | 12 | rs886125 | 8 |
| 109851249 | 12 | rs876311 | 8 |
| 109853784 | 12 | rs7953254 | 8 |
| 109861020 | 12 | rs7979656 | 8 |
| 109861304 | 12 | rs10849918 | 8 |
| 109861434 | 12 | rs10849920 | 8 |
| 109869679 | 12 | rs6489844 | 8 |
| 109870401 | 12 | rs2106408 | 8 |
| 109871050 | 12 | rs2157873 | 8 |
| 109876182 | 12 | rs3919447 | 8 |
| 109877902 | 12 | rs4766522 | 8 |
| 109878710 | 12 | rs2106407 | 8 |
| 109880802 | 12 | rs2339706 | 8 |
| 109880996 | 12 | rs4509829 | 8 |
| 109883562 | 12 | rs4766523 | 8 |
| 109883829 | 12 | rs4766524 | 8 |
| 109884154 | 12 | rs7961663 | 8 |
| 109885008 | 12 | rs4766438 | 8 |
| 109885219 | 12 | rs4766439 | 8 |
| 109885606 | 12 | rs4766440 | 8 |
| 109885699 | 12 | rs4766526 | 8 |
| 109889853 | 12 | rs4766527 | 8 |
| 109891219 | 12 | rs12814105 | 8 |
| 109896846 | 12 | rs11065784 | 8 |
| 109910998 | 12 | rs7968960 | 8 |
| 109988416 | 12 | rs4378452 | 8 |
| 110021205 | 12 | rs1001484 | 8 |
| 110030548 | 12 | rs10774613 | 8 |
| 110070809 | 12 | rs4766540 | 8 |
| 110072313 | 12 | rs10849927 | 8 |
| 110074875 | 12 | rs4766542 | 8 |
| 110082585 | 12 | rs756825 | 8 |
| 22672188 | 14 | rs8010887 | 9 |
| 22675813 | 14 | rs3783436 | 9 |
| 22677086 | 14 | rs1015089 | 9 |
| 22677570 | 14 | rs2013931 | 9 |
| 22677706 | 14 | rs999165 | 9 |
| 22682329 | 14 | rs2273394 | 9 |
| 22682426 | 14 | rs927041 | 9 |
| 22683680 | 14 | rs2331937 | 9 |
| 22687034 | 14 | rs12588118 | 9 |
| 22689742 | 14 | rs2268878 | 9 |
| 22698306 | 14 | rs4982736 | 9 |
| 22702346 | 14 | rs910795 | 9 |
| 22706597 | 14 | rs2268877 | 9 |
| 22706798 | 14 | rs2268876 | 9 |
| 22710634 | 14 | rs2268873 | 9 |
| 22713953 | 14 | rs6573011 | 9 |
| 22723028 | 14 | rs7141505 | 9 |
| 22726721 | 14 | rs761875 | 9 |
| 22734042 | 14 | rs12892307 | 9 |
| 22734486 | 14 | rs4982740 | 9 |
| 22768904 | 14 | rs178758 | 9 |
| 22776578 | 14 | rs178765 | 9 |
| 22778082 | 14 | rs178768 | 9 |
| 22784088 | 14 | rs178778 | 9 |
| 22784522 | 14 | rs17128091 | 9 |
| 22791597 | 14 | rs1955560 | 9 |
| 22791720 | 14 | rs11629105 | 9 |
| 22792037 | 14 | rs7160550 | 9 |
| 22792986 | 14 | rs12323394 | 9 |
| 22794987 | 14 | rs977870 | 9 |
| 22795079 | 14 | rs977871 | 9 |
| 22796375 | 14 | rs7157342 | 9 |
| 22799145 | 14 | rs6573081 | 9 |
| 22799242 | 14 | rs2144039 | 9 |
| 22799590 | 14 | rs2064483 | 9 |
| 22801931 | 14 | rs11625337 | 9 |
| 22802578 | 14 | rs1963151 | 9 |
| 22803565 | 14 | rs3811180 | 9 |
| 22804508 | 14 | rs6573084 | 9 |
| 22807362 | 14 | rs8017553 | 9 |
| 22810078 | 14 | rs4375573 | 9 |
| 22810827 | 14 | rs7141429 | 9 |
| 22812175 | 14 | rs8010663 | 9 |
| 22815373 | 14 | rs10131813 | 9 |
| 22816109 | 14 | rs1057119 | 9 |
| 22819435 | 14 | rs10144278 | 9 |
| 22824420 | 14 | rs17256211 | 9 |
| 22825438 | 14 | rs2295124 | 9 |
| 22846939 | 14 | rs2231301 | 9 |
| 22849474 | 14 | rs1535094 | 9 |
| 22865816 | 14 | rs2268330 | 9 |
| 22876030 | 14 | rs4270088 | 9 |
| 22882077 | 14 | rs12882406 | 9 |
| 22884409 | 14 | rs4982753 | 9 |
| 22885249 | 14 | rs10130118 | 9 |
| 22886838 | 14 | rs12896494 | 9 |
| 22893753 | 14 | rs7148564 | 9 |
| 22895553 | 14 | rs4671 | 9 |
| 22901017 | 14 | rs2284652 | 9 |
| 22906941 | 14 | rs3759614 | 9 |
| 22909873 | 14 | rs10137082 | 9 |
| 22911857 | 14 | rs7145531 | 9 |
| 22913203 | 14 | rs8014568 | 9 |
| 22914819 | 14 | rs4553531 | 9 |
| 22915084 | 14 | rs3811178 | 9 |
| 22918151 | 14 | rs723840 | 9 |
| 22923469 | 14 | rs8006357 | 9 |
| 22924164 | 14 | rs178638 | 9 |
| 22926736 | 14 | rs178642 | 9 |
| 22927191 | 14 | rs8022522 | 9 |
| 22931651 | 14 | rs365990 | 9 |
| 22933642 | 14 | rs445754 | 9 |
| 22935725 | 14 | rs452036 | 9 |
| 22938125 | 14 | rs439735 | 9 |
| 22942932 | 14 | rs388914 | 9 |
| 22943957 | 14 | rs440466 | 9 |
| 22950923 | 14 | rs7143356 | 9 |
| 22952026 | 14 | rs7149517 | 9 |
| 22952695 | 14 | rs2331979 | 9 |
| 22953024 | 14 | rs3729833 | 9 |
| 22953579 | 14 | rs765020 | 9 |
| 22957485 | 14 | rs7159367 | 9 |
| 22957880 | 14 | rs12894524 | 9 |
| 22958505 | 14 | rs2277475 | 9 |
| 22960822 | 14 | rs743567 | 9 |
| 22962728 | 14 | rs7157716 | 9 |
| 22967306 | 14 | rs1951154 | 9 |
| 22968867 | 14 | rs735711 | 9 |
| 22972593 | 14 | rs2069540 | 9 |
| 22973220 | 14 | rs2239578 | 9 |
| 22976706 | 14 | rs1317972 | 9 |
| 22977448 | 14 | rs875908 | 9 |
| 22979402 | 14 | rs8005199 | 9 |
| 22981247 | 14 | rs11621360 | 9 |
| 22985782 | 14 | rs10873105 | 9 |
| 23009921 | 14 | rs1028587 | 9 |
| 23010199 | 14 | rs1028589 | 9 |
| 23013758 | 14 | rs7154379 | 9 |
| 23014354 | 14 | rs2236261 | 9 |
| 23015206 | 14 | rs2295706 | 9 |
| 23017342 | 14 | rs7158784 | 9 |
| 23023607 | 14 | rs4982761 | 9 |
| 23031442 | 14 | rs7151583 | 9 |
| 23034259 | 14 | rs7149201 | 9 |
| 23046850 | 14 | rs223116 | 9 |
| 23048673 | 14 | rs69931 | 9 |
| 23049394 | 14 | rs1154175 | 9 |
| 23053169 | 14 | rs727926 | 9 |
| 23055813 | 14 | rs222671 | 9 |
| 23057830 | 14 | rs222674 | 9 |
| 23060945 | 14 | rs223122 | 9 |
| 23064357 | 14 | rs4982766 | 9 |
| 23066612 | 14 | rs8016202 | 9 |
| 23067853 | 14 | rs3825581 | 9 |
| 23069146 | 14 | rs3742488 | 9 |
| 23072727 | 14 | rs3742489 | 9 |
| 23084051 | 14 | rs3783439 | 9 |
| 23085741 | 14 | rs7144469 | 9 |
| 23086206 | 14 | rs7145877 | 9 |
| 23087118 | 14 | rs17184212 | 9 |
| 23088178 | 14 | rs222682 | 9 |
| 23111464 | 14 | rs11844366 | 9 |
| 23129120 | 14 | rs222732 | 9 |
| 23138577 | 14 | rs222696 | 9 |
| 23138737 | 14 | rs418012 | 9 |
| 23141800 | 14 | rs10140620 | 9 |
| 23158855 | 14 | rs222716 | 9 |
| 23168471 | 14 | rs222723 | 9 |
| 23169038 | 14 | rs222726 | 9 |
| 23169156 | 14 | rs222727 | 9 |
| 23169408 | 14 | rs222729 | 9 |
| 23185313 | 14 | rs17199254 | 9 |
| 23185949 | 14 | rs17184233 | 9 |
| 23190110 | 14 | rs222616 | 9 |
| 23192507 | 14 | rs4145675 | 9 |
| 23195726 | 14 | rs222626 | 9 |
| 23196452 | 14 | rs222629 | 9 |
| 23197431 | 14 | rs222631 | 9 |
| 23199414 | 14 | rs11626467 | 9 |
| 23199979 | 14 | rs221708 | 9 |
| 23202628 | 14 | rs221706 | 9 |
| 23202771 | 14 | rs221705 | 9 |
| 23203751 | 14 | rs221704 | 9 |
| 23204791 | 14 | rs12887072 | 9 |
| 23206994 | 14 | rs1438123 | 9 |
| 23207192 | 14 | rs7140616 | 9 |
| 23207427 | 14 | rs7141117 | 9 |
| 23207992 | 14 | rs1885593 | 9 |
| 23209847 | 14 | rs1866226 | 9 |
| 23211168 | 14 | rs1535495 | 9 |
| 23211295 | 14 | rs17794525 | 9 |
| 23212756 | 14 | rs977492 | 9 |
| 23217448 | 14 | rs2840251 | 9 |
| 23217569 | 14 | rs1015731 | 9 |
| 60888291 | 15 | rs1553107 | 10 |
| 60898001 | 15 | rs12594810 | 10 |
| 60914827 | 15 | rs2242379 | 10 |
| 60918821 | 15 | rs937419 | 10 |
| 60931289 | 15 | rs11071694 | 10 |
| 60932827 | 15 | rs1873151 | 10 |
| 60933863 | 15 | rs8025643 | 10 |
| 60936208 | 15 | rs580621 | 10 |
| 60936636 | 15 | rs671866 | 10 |
| 60938016 | 15 | rs655457 | 10 |
| 60945482 | 15 | rs557775 | 10 |
| 60945881 | 15 | rs561428 | 10 |
| 60946192 | 15 | rs2220476 | 10 |
| 60947757 | 15 | rs4775554 | 10 |
| 60948753 | 15 | rs17815421 | 10 |
| 60949580 | 15 | rs595866 | 10 |
| 60951526 | 15 | rs674109 | 10 |
| 60952139 | 15 | rs565375 | 10 |
| 60952673 | 15 | rs16945982 | 10 |
| 60954316 | 15 | rs12443298 | 10 |
| 60954537 | 15 | rs4775555 | 10 |
| 60956845 | 15 | rs12441248 | 10 |
| 60957022 | 15 | rs525431 | 10 |
| 60957227 | 15 | rs650716 | 10 |
| 60958642 | 15 | rs489667 | 10 |
| 60958931 | 15 | rs12440061 | 10 |
| 60959520 | 15 | rs570763 | 10 |
| 60961100 | 15 | rs11853643 | 10 |
| 60966349 | 15 | rs12914720 | 10 |
| 60968062 | 15 | rs11639162 | 10 |
| 60968639 | 15 | rs12443469 | 10 |
| 60969371 | 15 | rs1817732 | 10 |
| 60969687 | 15 | rs12903314 | 10 |
| 60970492 | 15 | rs729237 | 10 |
| 60970687 | 15 | rs729238 | 10 |
| 60974222 | 15 | rs779788 | 10 |
| 60977505 | 15 | rs673795 | 10 |
| 60978895 | 15 | rs729197 | 10 |
| 60979501 | 15 | rs4274387 | 10 |
| 60985990 | 15 | rs8030784 | 10 |
| 60987775 | 15 | rs867670 | 10 |
| 60988279 | 15 | rs902034 | 10 |
| 60990221 | 15 | rs12591327 | 10 |
| 60994393 | 15 | rs902023 | 10 |
| 60997450 | 15 | rs12900905 | 10 |
| 61006108 | 15 | rs731747 | 10 |
| 61011559 | 15 | rs6494371 | 10 |
| 61014657 | 15 | rs12907431 | 10 |
| 61018089 | 15 | rs12591202 | 10 |
| 61021102 | 15 | rs1844292 | 10 |
| 61025461 | 15 | rs4774459 | 10 |
| 61028923 | 15 | rs11071702 | 10 |
| 61029336 | 15 | rs923424 | 10 |
| 61029884 | 15 | rs1994533 | 10 |
| 61030605 | 15 | rs4775567 | 10 |
| 61035998 | 15 | rs8034321 | 10 |
| 61045411 | 15 | rs6494380 | 10 |
| 61051291 | 15 | rs4775575 | 10 |
| 61052232 | 15 | rs12708475 | 10 |
| 61052448 | 15 | rs4238368 | 10 |
| 61054989 | 15 | rs4775582 | 10 |
| 61056153 | 15 | rs4775586 | 10 |
| 61057210 | 15 | rs4775587 | 10 |
| 61057386 | 15 | rs17751112 | 10 |
| 61059703 | 15 | rs7179788 | 10 |
| 61060189 | 15 | rs11639273 | 10 |
| 61062778 | 15 | rs4774463 | 10 |
| 61068573 | 15 | rs1489317 | 10 |
| 61076217 | 15 | rs11634442 | 10 |
| 61077038 | 15 | rs8026456 | 10 |
| 61077332 | 15 | rs11071708 | 10 |
| 61077423 | 15 | rs12913459 | 10 |
| 61079101 | 15 | rs11071709 | 10 |
| 61079499 | 15 | rs1489315 | 10 |
| 61080234 | 15 | rs12911467 | 10 |
| 61087533 | 15 | rs4775598 | 10 |
| 61089183 | 15 | rs16946249 | 10 |
| 61089362 | 15 | rs16946255 | 10 |
| 61098478 | 15 | rs4774467 | 10 |
| 61099481 | 15 | rs1873149 | 10 |
| 61100719 | 15 | rs12443295 | 10 |
| 61107025 | 15 | rs8039583 | 10 |
| 61107561 | 15 | rs8039242 | 10 |
| 61108492 | 15 | rs4514628 | 10 |
| 61108952 | 15 | rs1826040 | 10 |
| 61110832 | 15 | rs12324180 | 10 |
| 61110933 | 15 | rs4775604 | 10 |
| 61111101 | 15 | rs4775605 | 10 |
| 61111554 | 15 | rs16946330 | 10 |
| 61114658 | 15 | rs4775606 | 10 |
| 61115060 | 15 | rs4775607 | 10 |
| 61115351 | 15 | rs9806533 | 10 |
| 61120777 | 15 | rs3809566 | 10 |
| 61127280 | 15 | rs4075583 | 10 |
| 61130371 | 15 | rs3803499 | 10 |
| 61132579 | 15 | rs6494387 | 10 |
| 61135140 | 15 | rs16953475 | 10 |
| 61138740 | 15 | rs4775614 | 10 |
| 61139145 | 15 | rs3803501 | 10 |
| 61140813 | 15 | rs4774472 | 10 |
| 61142392 | 15 | rs12148828 | 10 |
| 61146617 | 15 | rs4619348 | 10 |
| 61147900 | 15 | rs1972041 | 10 |
| 61150954 | 15 | rs6738 | 10 |
| 61153335 | 15 | rs2937859 | 10 |
| 61156624 | 15 | rs2937857 | 10 |
| 61161309 | 15 | rs2652804 | 10 |
| 61166645 | 15 | rs11857703 | 10 |
| 61192419 | 15 | rs2652813 | 10 |
| 61195232 | 15 | rs4775622 | 10 |
| 61216139 | 15 | rs1126308 | 10 |
| 61221816 | 15 | rs4774478 | 10 |
| 61226239 | 15 | rs7162825 | 10 |
| 61226456 | 15 | rs7163254 | 10 |
| 61227991 | 15 | rs6494394 | 10 |
| 61256899 | 15 | rs2588859 | 10 |
| 61258386 | 15 | rs2588860 | 10 |
| 61261444 | 15 | rs920864 | 10 |
| 61271030 | 15 | rs2729793 | 10 |
| 61272580 | 15 | rs1910094 | 10 |
| 61275301 | 15 | rs8032253 | 10 |
| 61303319 | 15 | rs17828874 | 10 |
| 61303812 | 15 | rs8037296 | 10 |
| 61313292 | 15 | rs11858666 | 10 |
| 61328461 | 15 | rs1992620 | 10 |
| 61331159 | 15 | rs1444405 | 10 |
| 61341047 | 15 | rs1017545 | 10 |
| 61367208 | 15 | rs11071738 | 10 |
| 61367649 | 15 | rs7176591 | 10 |
| 61377025 | 15 | rs17762576 | 10 |
| 61385483 | 15 | rs745338 | 10 |
| 61390539 | 15 | rs10851727 | 10 |
| 60107428 | 19 | rs4806460 | 11 |
| 60120271 | 19 | rs1671160 | 11 |
| 60126270 | 19 | rs622941 | 11 |
| 60126387 | 19 | rs623383 | 11 |
| 60126630 | 19 | rs634742 | 11 |
| 60130978 | 19 | rs269933 | 11 |
| 60131633 | 19 | rs269938 | 11 |
| 60132169 | 19 | rs269940 | 11 |
| 60132697 | 19 | rs269941 | 11 |
| 60132880 | 19 | rs269942 | 11 |
| 60133714 | 19 | rs269950 | 11 |
| 60133807 | 19 | rs269951 | 11 |
| 60135236 | 19 | rs269955 | 11 |
| 60136986 | 19 | rs269957 | 11 |
| 60139313 | 19 | rs7359934 | 11 |
| 60139407 | 19 | rs7359929 | 11 |
| 60139652 | 19 | rs4806626 | 11 |
| 60141567 | 19 | rs775879 | 11 |
| 60143609 | 19 | rs775883 | 11 |
| 60144946 | 19 | rs775886 | 11 |
| 60173132 | 19 | rs703465 | 11 |
| 60186552 | 19 | rs10412915 | 11 |
| 60186693 | 19 | rs11672113 | 11 |
| 60189655 | 19 | rs1036231 | 11 |
| 60190556 | 19 | rs7253480 | 11 |
| 60193391 | 19 | rs1654495 | 11 |
| 60193646 | 19 | rs1654496 | 11 |
| 60194422 | 19 | rs1671225 | 11 |
| 60195599 | 19 | rs1654498 | 11 |
| 60197083 | 19 | rs1654503 | 11 |
| 60198861 | 19 | rs1671133 | 11 |
| 60201677 | 19 | rs11667105 | 11 |
| 60203949 | 19 | rs12768 | 11 |
| 60204044 | 19 | rs1043673 | 11 |
| 60204128 | 19 | rs1043678 | 11 |
| 60204243 | 19 | rs1043684 | 11 |
| 60207784 | 19 | rs10415310 | 11 |
| 60220912 | 19 | rs12462907 | 11 |
| 60221350 | 19 | rs7257207 | 11 |
| 60240370 | 19 | rs4806636 | 11 |
| 60241570 | 19 | rs1654431 | 11 |
| 60244635 | 19 | rs1671214 | 11 |
| 60245888 | 19 | rs1671216 | 11 |
| 60245950 | 19 | rs8113032 | 11 |
| 60246211 | 19 | rs1671218 | 11 |
| 60251552 | 19 | rs1059211 | 11 |
| 60262594 | 19 | rs1671176 | 11 |
| 60270514 | 19 | rs1654466 | 11 |
| 60273323 | 19 | rs1654467 | 11 |
| 60280223 | 19 | rs1627029 | 11 |
| 60280303 | 19 | rs1626809 | 11 |
| 60285891 | 19 | rs2257933 | 11 |
| 60289181 | 19 | rs2290476 | 11 |
| 60306735 | 19 | rs2532060 | 11 |
| 60315618 | 19 | rs604216 | 11 |
| 60363186 | 19 | rs891187 | 11 |
| 60374715 | 19 | rs16986289 | 11 |
| 60387556 | 19 | rs8106715 | 11 |
| 60400369 | 19 | rs2288521 | 11 |
| 60405347 | 19 | rs2288515 | 11 |
| 60407131 | 19 | rs9304763 | 11 |
| 60408612 | 19 | rs2288516 | 11 |
| 60413053 | 19 | rs876515 | 11 |
| 60418032 | 19 | rs10419994 | 11 |
| 60426844 | 19 | rs8103705 | 11 |
| 60435737 | 19 | rs13343928 | 11 |
| 60448792 | 19 | rs3745920 | 11 |
| 60461266 | 19 | rs4283339 | 11 |
| 60465402 | 19 | rs10403164 | 11 |
| 60479681 | 19 | rs3786544 | 11 |
| 60491730 | 19 | rs16960290 | 11 |
| 60495632 | 19 | rs2607336 | 11 |
| 60511657 | 19 | rs1172822 | 11 |
| 60516446 | 19 | rs4806660 | 11 |
| 60523000 | 19 | rs2384687 | 11 |
| 60524239 | 19 | rs10412726 | 11 |
| 60524367 | 19 | rs10411773 | 11 |
| 60525272 | 19 | rs11668309 | 11 |
| 60525476 | 19 | rs11668344 | 11 |
| 60525997 | 19 | rs8113016 | 11 |
| 60526260 | 19 | rs10425848 | 11 |
| 60537198 | 19 | rs7253685 | 11 |
| 60542081 | 19 | rs1870073 | 11 |
| 60546125 | 19 | rs2384685 | 11 |
| 60551251 | 19 | rs1058511 | 11 |
| 60557339 | 19 | rs11084396 | 11 |
| 60562163 | 19 | rs12976922 | 11 |
| 60564003 | 19 | rs897799 | 11 |
| 60568923 | 19 | rs1042506 | 11 |
| 60571684 | 19 | rs1126757 | 11 |
| 60575691 | 19 | rs6509940 | 11 |
| 60589139 | 19 | rs17700376 | 11 |
| 60592229 | 19 | rs11668142 | 11 |
| 29642688 | 20 | rs750144 | 12 |
| 29646930 | 20 | rs6060171 | 12 |
| 29656050 | 20 | rs6058189 | 12 |
| 29689201 | 20 | rs6119610 | 12 |
| 29695298 | 20 | rs6060446 | 12 |
| 29698963 | 20 | rs6058259 | 12 |
| 29712464 | 20 | rs6060563 | 12 |
| 29718434 | 20 | rs6121038 | 12 |
| 29722475 | 20 | rs10439608 | 12 |
| 29729111 | 20 | rs12479491 | 12 |
| 29730506 | 20 | rs6060652 | 12 |
| 29745884 | 20 | rs6060763 | 12 |
| 29750989 | 20 | rs1994251 | 12 |
| 29763883 | 20 | rs6060857 | 12 |
| 29766960 | 20 | rs6060870 | 12 |
| 29769636 | 20 | rs1484994 | 12 |
| 29770860 | 20 | rs3181073 | 12 |
| 29789434 | 20 | rs6089056 | 12 |
| 29795583 | 20 | rs6060919 | 12 |
| 29802806 | 20 | rs2149282 | 12 |
| 29811493 | 20 | rs6060930 | 12 |
| 29817803 | 20 | rs6087778 | 12 |
| 29826732 | 20 | rs6058460 | 12 |
| 29835775 | 20 | rs6060944 | 12 |
| 29841692 | 20 | rs6060950 | 12 |
| 29845769 | 20 | rs6060952 | 12 |
| 29849218 | 20 | rs6119725 | 12 |
| 29851653 | 20 | rs1075698 | 12 |
| 29853708 | 20 | rs6060959 | 12 |
| 29861352 | 20 | rs6058465 | 12 |
| 29866163 | 20 | rs2377317 | 12 |
| 29872241 | 20 | rs4911532 | 12 |
| 29874538 | 20 | rs6121242 | 12 |
| 29877130 | 20 | rs1887732 | 12 |
| 29878755 | 20 | rs6121243 | 12 |
| 29882712 | 20 | rs4518038 | 12 |
| 29885120 | 20 | rs6089093 | 12 |
| 29893424 | 20 | rs6060989 | 12 |
| 29899749 | 20 | rs8118150 | 12 |
| 29901183 | 20 | rs3746599 | 12 |
| 29907134 | 20 | rs4911533 | 12 |
| 29910233 | 20 | rs4911534 | 12 |
| 29915505 | 20 | rs6087786 | 12 |
| 29915753 | 20 | rs947311 | 12 |
| 29923100 | 20 | rs8120234 | 12 |
| 29931947 | 20 | rs6058480 | 12 |
| 29936849 | 20 | rs6061015 | 12 |
| 29941877 | 20 | rs11699897 | 12 |
| 29942901 | 20 | rs6061020 | 12 |
| 29953331 | 20 | rs2103656 | 12 |
| 29953523 | 20 | rs2092363 | 12 |
| 29957645 | 20 | rs1883506 | 12 |
| 29966327 | 20 | rs2143208 | 12 |
| 29968191 | 20 | rs8121840 | 12 |
| 29972121 | 20 | rs4911538 | 12 |
| 29977822 | 20 | rs6089119 | 12 |
| 29980707 | 20 | rs4243978 | 12 |
| 29982229 | 20 | rs6061036 | 12 |
| 29990726 | 20 | rs6061043 | 12 |
| 29995704 | 20 | rs6061045 | 12 |
| 29997610 | 20 | rs6089127 | 12 |
| 30005104 | 20 | rs1883507 | 12 |
| 30008057 | 20 | rs6061052 | 12 |
| 30013187 | 20 | rs6061059 | 12 |
| 30015761 | 20 | rs6061062 | 12 |
| 30026261 | 20 | rs6061069 | 12 |
| 30031338 | 20 | rs6121302 | 12 |
| 30033526 | 20 | rs754568 | 12 |
| 30039143 | 20 | rs5028704 | 12 |
| 30042128 | 20 | rs6089140 | 12 |
| 30042269 | 20 | rs6089141 | 12 |
| 30046842 | 20 | rs6061079 | 12 |
| 30047677 | 20 | rs6061080 | 12 |
| 30049423 | 20 | rs714604 | 12 |
| 30049685 | 20 | rs714605 | 12 |
| 30055015 | 20 | rs6061086 | 12 |
| 30055887 | 20 | rs6061087 | 12 |
| 30056075 | 20 | rs6061088 | 12 |
| 30059043 | 20 | rs6058511 | 12 |
| 30064328 | 20 | rs6061095 | 12 |
| 30071707 | 20 | rs6061104 | 12 |
| 30082326 | 20 | rs17093749 | 12 |
| 30082448 | 20 | rs6061112 | 12 |
| 30084293 | 20 | rs6121318 | 12 |
| 30092228 | 20 | rs6579318 | 12 |
| 30093703 | 20 | rs4561724 | 12 |
| 30109620 | 20 | rs6058522 | 12 |
| 30124958 | 20 | rs2297304 | 12 |
| 30125935 | 20 | rs6061154 | 12 |
| 30126466 | 20 | rs3746601 | 12 |
| 30128035 | 20 | rs242599 | 12 |
| 30130609 | 20 | rs242602 | 12 |
| 30130720 | 20 | rs242603 | 12 |
| 30135536 | 20 | rs242607 | 12 |

TABLE 9
To help identify the SNPs that belong to each
gene the following table can be used.

| | | Exclusion Analysis |
|---|---|---|
| Chromosome | Gene | Gene Number |
| 1 | TNNT2 | 1 |
| 2 | TTN | 2 |
| 3 | CAV3 | 3 |
| 3 | RAF1 | 4 |
| 3 | MYL3 | 5 |
| 7 | PRKAG2 | 6 |
| 11 | MYBPC3 | 7 |
| 12 | MYL2 | 8 |
| 14 | MYH6 + MYH7 | 9 |
| 15 | TPM1 | 10 |
| 19 | TNNI3 | 11 |
| 20 | MYLK2 | 12 |

Each gene was given a number to help with the analysis (as detailed in Table 8 above).

1. A method of excluding the involvement of a gene in a genetic disorder in a family, said method comprising:
(a) selecting a population of single nucleotide polymorphisms (SNPs) that
(i) are proximate to the gene; and
(ii) have a minor allele frequency sufficient to establish an appropriate level of heterozygosity;
(b) identifying said SNPs in nucleic acid samples provided by each of at least two subjects affected by the genetic disorder and linked by pedigree; and
wherein, the presence of SNP alleles which are oppositely homozygous in at least two of the affected subjects, indicates that the gene is unlikely to be involved in the genetic disorder.

2. The method of claim 1, wherein the genetic disorder is a dominant genetic disorder.

3. The method of claim 1, wherein the genetic disorder is selected from the group consisting of:
(i) Familial Breast cancer;
(ii) Hereditary haemorrhagic telangectasia;
(iii) Hereditary spastic paraplegia;
(iv) Cerebral cavernous malformations;
(v) Hypertrophic cardiomyopathy;
(vi) Dilated cardiomyopathy;
(vii) Long QT;
(viii) Adult polycystic kidney disease;
(ix) Tuberous sclerosis;
(x) Spinocerebellar ataxia;
(xi) Alzheimer's;
(xii) Marfan syndrome;
(xiii) Noonan syndrome;
(xiv) Dominant retinitis pigmentosa;
(xv) Multiple epiphyseal dysplasia;
(xvi) Ehlers Danlos;
(xvii) Hereditary colorectal cancer;
(xviii) Juvenile polyposis; and
(xix) Familial paraganglioma.

4. A method of determining whether or not a subject should be tested for the presence of mutations in a gene associated with or causative of, a genetic disorder, said method comprising:
(a) selecting a population of single nucleotide polymorphisms (SNPs) that
(i). are proximate to the gene; and
(ii). have a minor allele frequency sufficient to establish an appropriate level of heterozygosity;
(b) identifying the SNPs in nucleic acid samples provided by each of at least two subjects affected by the genetic disorder and linked by pedigree to each other and said subject; and
wherein, the presence of SNP alleles which are oppositely homozygous in at least two of said affected subjects, indicates that the subject need not be tested for mutations in the gene causative of, or associated with, the genetic disorder.

5. The method of claim 1, wherein the pedigree links that exist between the at least two affected subjects do not represent, constitute or comprise a parent/child link.

6. A kit comprising:
(a) oligonucleotide primers capable of hybridising upstream and down stream of nucleotide sequences comprising SNPs that:
(i) are proximate to the gene; and
(ii) have a minor allele frequency sufficient to establish an appropriate level of heterozygosity.

7. The kit of claim 6, wherein the oligonucleotide primers are capable of hybridising upstream and down stream of nucleotide sequences comprising the SNPs identified in Table 3 and/or Table 8.

8. A data set comprising information pertaining to SNPs that:
(i) are proximate to a gene causative of or associated with a genetic disorder; and
(ii) have a minor allele frequency sufficient to establish an appropriate level of heterozygosity.

9. The data set of claim 8, wherein the data set comprises the information contained in Table 3 and/or Table 8.

10. A method of excluding the involvement of the BRCA1 and/or BRCA2 genes in familial breast cancer in a family, said method comprising:
(a) selecting a population of single nucleotide polymorphisms (SNPs) that
(i) are within approximately 10 MB of the BRCA1 and/or BRCA2 genes; and
(ii) have a MAF value of greater than 0.1
(b) identifying said SNPs in nucleic acid samples provided by each of at least two subjects affected by familial breast cancer and linked by pedigree; and
wherein, the presence of SNP alleles which are oppositely homozygous in at least two of the affected subjects, indicates that the BRCA1 and/or BRCA2 genes are unlikely to be involved in the familial breast cancer present in said family.

11. The method of claim 10, wherein the SNPs are located within approximately 8 MB, 5 MB, 2 MB or 1 MB of the BRCA1 and/or BRCA2 genes.

12. A method of excluding the involvement of one or more genes in hypertrophic cardiomyopathy or dilated cardiomyopathy in a family, said method comprising:
(a) selecting a population of single nucleotide polymorphisms (SNPs) that
(i) are within approximately 15 MB of the gene(s); and
(ii) have a MAF value of greater than 0.1;
(b) identifying said SNPs in nucleic acid samples provided by each of at least two subjects affected by hypertrophic cardiomyopathy or dilated cardiomyopathy and linked by pedigree;
wherein, the presence of SNP alleles which are oppositely homozygous in at least two of the affected subjects, indicates that the gene(s) is/are unlikely to be involved in the hypertrophic cardiomyopathy or dilated cardiomyopathy present in said family.

13. The method of claim 12, wherein the SNPs are located within approximately 12 MB, 10 MB, 5 MB or 2.5 MB of the gene.

14. The method of claim 12, wherein the gene is selected from the group consisting of TTN, Original) MYH6/7, MYBPC3, RAF1, PRKAG2, TPM1, TNNT2, MYLK2, TNNI3, MYL3, MYL2 and CAV3.

15. The method of claim 4, wherein the pedigree links that exist between the at least two affected subjects do not represent, constitute or comprise a parent/child link.
